(19) United States
(12) Patent Application Publication
(10) Pub. No.: US 2009/0226929 A1
(43) Pub. Date: Sep. 10, 2009

(54) TITINIC ION CHANNEL, COMPOSITIONS AND METHODS OF USE

(75) Inventors: Magdalene M. Moran, Brookline, MA (US); Jayhong A. Chong, Brookline, MA (US)

Correspondence Address: ROPES & GRAY LLP, PATENT DOCKETING 39/41, ONE INTERNATIONAL PLACE, BOSTON, MA, 02110-2624, US

(73) Assignee: Hydra Biosciences, Inc, Cambridge, MA (US)

(21) Appl. No.: 12/225,051

(22) PCT Filed: Mar. 16, 2007
(86) PCT No.: PCT/US2007/006661

§ 371 Date: Jan. 21, 2009

Related U.S. Application Data
(60) Provisional application No. 60/783,281, filed on Mar. 16, 2006.

(51) Int. Cl. G01N 33/53; C07H 21/04; C12N 15/63; C12N 5/10; C12P 21/02; C07K 14/435; C12Q 1/02; C07K 16/00

(52) U.S. Cl. 435/7.1; 536/23.5; 435/320.1; 435/252.3; 435/358; 435/369; 435/368; 435/69.1; 530/350; 435/29; 530/387.9

(57) ABSTRACT

The present invention provides methods and compositions related to a novel voltage sensitive protein comprising four transmembrane domains.

### RELATED APPLICATIONS

This application claims the benefit of priority to United Stated provisional application Ser. No. 60/783,281, filed Mar. 16, 2006, the entire contents of which is hereby incorporated by reference in its entirety.

### BACKGROUND OF THE INVENTION

Voltage is a key regulator of cellular function. Changes in voltage affect numerous cellular processes. In addition to opening or closing voltage-dependent ion channels, recent work has also demonstrated that voltage-gated enzymes exist, some of which may also have channel activity.

A variety of voltage sensitive proteins exist to mediate ion flux across cellular membranes and to initiate signal transduction cascades. The proper expression and function of such proteins, as well as other membrane protein and ion channels, is essential for the maintenance of cell function, intracellular communication, and the like. Numerous diseases are the result of misregulation of membrane potential. Given the central importance of voltage sensitive proteins in modulating membrane potential and ion flux in cells, identification of agents that can promote or inhibit the function of particular voltage sensitive proteins are of great interest as research tools and as possible therapeutic agents.

### SUMMARY

The present invention provides a novel voltage sensitive surface protein referred to herein as titinic. Titinic proteins comprise four transmembrane domains, with a stretch of arginines in the fourth transmembrane domain characteristic of the voltage sensor domain in the voltage-gated potassium channel shaker. This voltage sensitive domain (VSD) is followed by a long C-terminal sequence that bears little homology to known proteins. Based on a group of residues in the third transmembrane domain and some distant similarity to human transmembrane phosphatases, titinic appears to be a voltage sensitive protein that localizes to the cell surface, particularly in cells of the dorsal root ganglia, brain, and spinal cord. Its ability to bind signaling molecules such as PKN1 in a voltage-dependent fashion indicates a role in central and peripheral nervous system conditions involving hyperexcitability of neurons including, for example, epilepsy, cerebral ischemic disease, and pain.

The present invention provides compositions comprising titinic nucleic acid and amino acid sequences. The present invention further provides various screening assays using a cell expressing a titinic voltage sensitive protein in the membrane. Given the function of membrane proteins in mediating cellular homeostasis, screening assays to identify and/or characterize compounds that agonize or antagonize the function of titinic are of significant use. Compounds identified as having the ability to modulate titinic activity would be useful for the treatment of central or peripheral nervous system conditions involving neuronal hyperexcitability, such as pain, epilepsy and cerebral ischemic disease. In addition, identifying compounds that modulate the enzymatic activity of titinic (e.g., ability to modulate phosphatase or kinase activity) would be of significant utility, as they would have profound affects on the activity of other ion channels including members of the Transient Receptor Potential family. Compounds that agonize or antagonize one or more functions of ion channels or other membrane protein, for example a titinic voltage sensitive surface protein, can be used in the development of therapeutics or can be used in the development of in vitro assays to study voltage-dependent protein function.

In a first aspect, the invention provides nucleic acids encoding a titinic protein. In one embodiment, the nucleic acid is an isolated or recombinantly produced nucleic acid. In one embodiment, the titinic protein is voltage sensitive protein comprising four transmembrane domains. In one embodiment, the titinic protein comprising four transmembrane domains has one or more of the following functions: mediates polarization state of neuronal cells, modulates the activity of protein kinases such as PKN1 (optionally in a cellular polarization state dependent manner), mediates membrane potential, mediates membrane voltage, and/or modulates enzymatic activity in a cell.

In one embodiment, the nucleic acid or isolated nucleic acid encoding the titinic protein comprises a nucleotide sequence that hybridizes under stringent conditions, including a wash step of 0.2×SSC at 65° C., to the complementary sequence of the nucleic acid sequence set for in SEQ ID NO: 1. In another embodiment, the nucleic acid comprises a nucleotide sequence set forth in SEQ ID NO: 1. In yet another embodiment, the nucleic acid comprises a nucleotide sequence at least 70%, 75%, 80%, 85%, 90%, 95%, 98%, 99%, or 100% identical to SEQ ID NO: 1.

In another embodiment, the isolated nucleic acid encodes a titinic protein that comprises an amino acid sequence at least 70%, 75%, 80%, 85%, 90%, 95%, 98%, 99%, or 100% identical to SEQ ID NO: 2. In one embodiment, the titinic protein is a voltage sensitive protein containing four transmembrane domains. In one embodiment, the titinic protein comprising four transmembrane domains has one or more of the following functions: mediates polarization state of neuronal cells (e.g., changes in membrane potential), modulates activity of protein kinases such as PKN1, mediates membrane potential, and mediates membrane voltage in a cell. In one embodiment, the titinic protein comprises a bioactive fragment of SEQ ID NO: 2, or a variant of SEQ ID NO: 2. Exemplary bioactive fragments retain one or more of the functional properties of the full length protein.

The invention also provides antibodies to titinic including monoclonal and polyclonal antibodies. Such antibodies may be used for therapeutic (neutralizing antibodies or chimeric antibodies) or diagnostic purposes as well as research tools.

In one embodiment of any of the foregoing, the invention provides a composition comprising any of the foregoing nucleic acids. In one embodiment, the composition further comprises a heterologous nucleic acid sequence. The heterologous nucleic acid sequence is optionally operably linked to a nucleic acid comprising a titinic protein.

In one embodiment, the invention provides an expression vector comprising any of the foregoing nucleic acids or nucleic acid compositions. In one embodiment, the expression vector can replicate in at least one of a prokaryotic cell or a eukaryotic cell.

In one embodiment, the invention provides a host cell transfected with an expression vector of the invention. In one embodiment, the host cell is a prokaryotic cell, such as a bacterial cell. In another embodiment, the host cell is a eukaryotic cell, such as a primary or immortalized cell or cell line. Other exemplary eukaryotic cells include CHO cells, HEK cells, neuronal cells (e.g., neurons or glia), and microglia cells. In certain embodiments, the host cell is stably or transiently transfected with a nucleic acid encoding a titinic protein. In any of the foregoing, the invention contemplates that a host cell expressing a titinic protein expresses the protein in the membrane, and further that the expressed protein retains one or more of the functions of native titinic protein. Eukaryotic cells can be from any species including, but not limited to, yeast, insect, chick, fish, frog, mouse, rat, cat, dog, rabbit, cow, pig, horse, non-human primate, and human.

In another aspect, the invention provides a method for producing a recombinant titinic protein. The method comprises culturing a host cell expressing a nucleic acid encoding a titinic protein, expressing the titinic protein, and isolating such protein from the cell culture. For this aspect of the invention, one of skill in the art can select the appropriate cell type and species.

In a third aspect, the invention provides isolated or recombinantly produced titinic proteins. In one embodiment, the titinic protein is a voltage sensitive protein comprising four transmembrane domains. In one embodiment, the titinic protein comprising four transmembrane domains has one or more of the following functions: mediates polarization state of neuronal cells, modulates activity of protein kinases such as PKN1, mediates membrane potential, and mediates membrane voltage. Titinic proteins according to the present invention comprises an amino acid sequence at least 70%, 75%, 80%, 85%, 90%, 95%, 98%, 99%, or 100% identical to SEQ ID NO: 2. Exemplary titinic proteins comprise four transmembrane domains and retain one or more of the following functions: mediates polarization state of neuronal cells, modulates activity of protein kinases such as PKN1, mediates membrane potential, mediates membrane voltage, and/or modulates phosphatase activity in a cell.

In one embodiment, the titinic protein comprises a bioactive fragment of SEQ ID NO: 2, or a variant of SEQ ID NO: 2. Exemplary bioactive fragments retain one or more of the functional properties of the full length protein.

In one embodiment, the isolated or recombinantly produced titinic protein comprises an amino acid sequence encoded by a nucleic acid sequence that hybridizes under stringent conditions, including a wash step of 0.2×SSC at 65° C., to a sequence complementary to the sequence of SEQ ID NO: 1. In one embodiment, the titinic protein comprises an amino acid sequence encoded by a nucleic acid sequence that is at least 70%, 75%, 80%, 85%, 90%, 95%, 98%, 99%, or 100% identical to SEQ ID NO: 1. In one embodiment the titinic protein encoded by any of the foregoing nucleic acid sequences is a titinic voltage sensitive protein comprising four transmembrane domains and retaining one or more of the following biological functions: mediates polarization state of neuronal cells, modulates activity of protein kinases such as PKN1, mediates membrane potential, and mediates membrane voltage in a cell.

In one embodiment of any of the foregoing, the invention provides a composition comprising a titinic protein according to the present invention. In certain embodiments, the composition is a pharmaceutical composition formulated in a pharmaceutically acceptable carrier or excipient.

Methods for identifying compounds that modulate titinic activity are also provided herein. Compounds identified by such methods are useful for treating central or peripheral nervous system conditions involving hyperexcitability of neurons (e.g., epilepsy, pain, stroke, or cerebral ischemic conditions).

In one example, the present invention provides a method of screening for compounds that modulate an activity of a titinic protein. In one embodiment, the method of screening comprises providing a cell expressing a titinic protein. Such a cell is contacted with a candidate compound. Following contacting the cell with a candidate compound, a change in the activity (increase or decrease) of the titinic protein relative to a control cell that has not been contacted with the candidate compound identifies the candidate compounds as a compound being useful for treating central or peripheral conditions involving hyperexcitability of neurons. Titinic activities include, for example, changes in polarization of neuronal cells, changes in intracellular ion concentration, cytoskeletal rearrangement, changes in kinase or phosphatase activity, or changes in pain phenotypes. A change in titinic activity also includes a modulation in the activity of proteins that are part of its signaling pathway. Thus, a change in titinic activity includes a change in PKN1 activity, for example. In all foregoing aspects of the invention, the protein that interacts with titinic such as the PKN1 protein may be a fusion protein, such that the promoter and/or all or part of a titinic interacting protein (e.g., PKN1) coding region is operably linked to a second, heterologous nucleic acid sequence. Optionally, the second, heterologous nucleic acid sequence is a reporter gene, that is, a gene whose expression may be assayed; reporter genes include, without limitation, those encoding glucuronidase (GUS), luciferase, chloramphenicol transacetylase (CAT), green fluorescent protein (GFP), alkaline phosphatase, and .beta.-galactosidase. Accordingly, a change in PKN1 activity for example may be measured by a change in reporter gene activity.

In all foregoing aspects of the invention, the titinic protein may be co-expressed with other proteins including ion channels such as TRP channels. Exemplary channels include TRPV1, Hv1, TRPV3, TRPC3, GIRK (GIRX1, 2, 3, or 4), IRK1, TRPC6, TRPC6, and CaV1.2. Compounds having the ability to modulate titinic activity are identified by their ability to modulate such ion channels in a voltage dependent fashion.

In one embodiment, the cell expresses a titinic protein comprising four transmembrane domains and encoded by a nucleic acid that hybridizes under stringent conditions, including a wash step of 0.2×SSC at 65° C., to a nucleic acid sequence set forth in SEQ ID NO: 1. In another embodiment, the cell expresses a titinic protein according to any of the foregoing aspects or embodiments of the invention. The cell may express the titinic protein endogenously or titinic protein may be supplied or supplemented by exogenously expressing the protein in the cell In one embodiment, the cell comprises a vector comprising a nucleic acid sequence encoding a titinic protein.

In one embodiment, the cell is a prokaryotic cell or a eukaryotic cell. In another embodiment, the prokaryotic cell is a bacterial cell. In another embodiment, the eukaryotic cell is a primary cell or an immortalized cell or cell line. Other exemplary eukaryotic cells include CHO cells, HEK cells, neuronal cells (e.g., neurons or glia), and microglia cells. In certain embodiments, the host cell is stably or transiently transfected with a nucleic acid encoding a titinic protein. In any of the foregoing, the invention contemplates that a host cell expressing a titinic protein expresses the protein in the membrane, and further that the expressed protein retains one or more of the functions of native titinic protein. Eukaryotic cells can be from any species including, but not limited to, yeast, chick, fish, Fog, mouse, rat, cat, dog, rabbit, cow, pig, horse, non-human primate, and human.

In one embodiment, the compound agonizes an activity (e.g., one or more functions) of the titinic protein. In another embodiment, the compound antagonizes an activity (e.g., one or more functions) of the titinic protein.

In one embodiment, the compound binds to the titinic protein or a PKN1 protein.

In one embodiment, the compound promotes changes in polarization of neuronal cells (e.g., via effects on other ion channels) or changes in intracellular ion concentration, cytoskeletal rearrangement, kinase or phosphatase activity, or pain phenotypes. A change in titinic activity also includes a modulation in the activity of proteins that are part of its signaling pathway. Thus, a change in titinic activity includes a change in PKN1 activity, for example.

In one embodiment of any of the foregoing, the compound can be any of a nucleic acid, a protein, or a small molecule. Exemplary nucleic acids include, but are not limited to, RNAi constructs, antisense oligonucleotides, and ribozymes. Exemplary proteins include, but are not limited to, antibodies. Exemplary small molecules have a molecular weight of less than approximately 600 daltons.

Candidate compounds according to any of the foregoing embodiments of this aspect of the invention, can be screened individually, in pools of more than one compounds, or by screening libraries of compounds. Furthermore, candidate compounds can be screened in single cells or in a culture of cells comprising more than one cell. Screening can optionally be by a high-throughput format.

In a fifth aspect, the invention provides a method of screening for compounds that modulate an activity of a titinic protein. The method comprises providing a cell expressing a titinic protein. The cell expressing the titinic protein is contacted with a candidate compound. A change in ion flux of hydrogen ions is detected in the presence of the candidate compound versus the absence of the candidate compound.

A compound that promotes a change (increase or decrease; into or out of the cell) in ion flux is a compound that modulates an activity of a titinic protein.

In one embodiment, the cell expresses a titinic protein comprising four transmembrane domains and encoded by a nucleic acid that hybridizes under stringent conditions, including a wash step of 0.2×SSC at 65° C., to a nucleic acid sequence that is complementary to the sequence set forth in SEQ ID NO: 1. In another embodiment, the cell expresses a titinic protein according to any of the foregoing aspects or embodiments of the invention. The cell may express the titinic protein endogenously or titinic protein may be supplied or supplemented by exogenously expressing the protein in the cell. In one embodiment, the cell comprises a vector comprising a nucleic acid sequence encoding a titinic protein.

In one embodiment, the cell is a prokaryotic cell or a eukaryotic cell. In another embodiment, the prokaryotic cell is a bacterial cell. In another embodiment, the eukaryotic cell is a primary cell or an immortalized cell or cell line. Other exemplary eukaryotic cells include CHO cells, HEK cells, neuronal cells (e.g., neurons or glia), and microglia cells. In certain embodiments, the host cell is stably transfected with a nucleic acid encoding a titinic protein. In any of the foregoing, the invention contemplates that a host cell expressing a titinic protein expresses the protein in the membrane, and further that the expressed protein retains one or more of the functions of native titinic protein. Eukaryotic cells can be from any species include, but not limited to, yeast, chick, fish, frog, mouse, rat, cat, dog, rabbit, cow, pig, horse, non-human primate, and human.

In one embodiment, the compound agonizes an activity (e.g., one or more function) of the titinic protein. In another embodiment, the compound antagonizes an activity (e.g., one or more function) of the titinic protein.

In one embodiment, the compound binds to the titinic protein.

In one embodiment, the compound promotes a change in the polarization state of a neuronal cell, a change in intracellular ion concentration, a cytoskeletal rearrangement, kinase or phosphatase activity, or change in a pain phenotype. The compound may also modulate the activity of proteins that are part of its signaling pathway including, for example, a change in PKN1 activity. In another embodiment, the compound promotes a change in ion flux, and the change is an increase or decrease in ion flux. In another embodiment, the compound, whether it increases or decreases ion flux, also promotes a change in the direction of ion movement across the cell membrane.

For any of the foregoing, the change in ion flux can be detected by standard methods known in the art such as patch clamp, fluorescent membrane potential assays, pH sensitive assays, or calcium flux assays (e.g., fluorescent or radioactive).

In one embodiment of any of the foregoing, the compound can be any of a nucleic acid, a protein, or a small molecule. Exemplary nucleic acids include, but are not limited to, RNAi constructs, antisense oligonucleotides, and ribozymes. Exemplary proteins include, but are not limited to, antibodies. Exemplary small molecules have a molecular weight of less than approximately 600 daltons.

Candidate compounds according to any of the foregoing embodiments of this aspect of the invention, can be screened individually, in pools of more than one compounds, or by screening libraries of compounds. Furthermore, candidate compounds can be screened in single cells or in a culture of cells comprising more than one cell. Screening can optionally be by a high-throughput format.

In a sixth aspect, the invention provides a method of screening for compounds that modulate an activity of a titinic protein. The method comprises providing a neuronal cell expressing a titinic protein. The cell is contacted with a candidate compound. A change in production of superoxide ions in said cell in the presence of said compound versus the absence of said compound is detected. A compound that promotes a change (increase or decrease) in production of superoxide ions is a compound that modulates an activity of a titinic protein.

In one embodiment, the cell expresses a titinic protein comprising four transmembrane domains and encoded by a nucleic acid that hybridizes under stringent conditions, including a wash step of 0.2×SSC at 65° C., to a nucleic acid sequence that is complementary to the sequence set forth in SEQ ID NO: 1. In another embodiment, the cell expresses a titinic protein according to any of the foregoing aspects or embodiments of the invention. The cell may express the titinic protein endogenously or titinic protein may be supplied or supplemented by exogenously expressing the protein in the cell. In one embodiment, the cell comprises a vector comprising a nucleic acid sequence encoding a titinic protein.

In certain embodiments, the host cell is stably transfected with a nucleic acid encoding a titinic protein. In any of the foregoing, the invention contemplates that a host cell expressing a titinic protein expresses the protein in the membrane, and further that the expressed protein retains one or more of the functions of native titinic protein. Cells can be from any species including, but not limited to, yeast, chick, fish, frog, mouse, rat, cat, dog, rabbit, cow, pig, horse, non-human primate, and human.

In one embodiment, the compound agonizes an activity (e.g., one or more function) of the titinic protein. In another embodiment, the compound antagonizes an activity (e.g., one or more function) of the titinic protein.

In one embodiment, the compound binds to the titinic protein.

In one embodiment of any of the foregoing, the compound can be any of a nucleic acid, a protein, or a small molecule. Exemplary nucleic acids include, but are not limited to, RNAi constructs, antisense oligonucleotides, and ribozymes. Exemplary proteins include, but are not limited to, antibodies. Exemplary small molecules have a molecular weight of less than approximately 600 daltons.

Candidate compounds according to any of the foregoing embodiments of this aspect of the invention, can be screened individually, in pools of more than one compounds, or by screening libraries of compounds. Furthermore, candidate compounds can be screened in single cells or in a culture of cells comprising more than one cell. Screening can optionally be by a high-throughput format.

In a seventh aspect, the invention provides a method of screening for titinic modulators, which are in turn useful for the treatment of central and peripheral nervous system conditions that involve hyperexcitability of neurons. The method comprises combining a candidate bioactive agent with a cell expressing a titinic protein and a PKN1 protein. A change in the activity of the PKN1 protein following the addition of a compound relative to a control cell (e.g., expressing only PKN1 protein but not titinic) identifies such compound as being a titinic modulator. A change in PKN1 activity may be determined by any standard method and includes, for example, a change in the amount of PKN1 recruited to the plasma membrane, a change in PKN1 signaling activity, a change in PKN1 phosphorylation levels.

The invention further provides a screening method that involves contacting a candidate compound with a PKN1 and titinic protein. The contacting event may occur inside a cell or in cell-free conditions. A candidate compound that modulates the binding of PKN1 and titinic protein is identified as a compound having the ability to modulate PKN1 activity. A compound having the ability to reduce the ability of titinic to bind PKN1 is identified as a compound useful for treating a central or peripheral nervous system condition involving hyperexcitability of neurons.

In one embodiment, the cell expresses a titinic protein comprising four transmembrane domains and encoded by a nucleic acid that hybridizes under stringent conditions, including a wash step of 0.2×SSC at 65° C., to a nucleic acid sequence that is complementary to the sequence set forth in SEQ ID NO: 1. In another embodiment, the cell expresses a titinic protein according to any of the foregoing aspects or embodiments of the invention. The cell may express the titinic protein endogenously or titinic protein may be supplied or supplemented by exogenously expressing the protein in the cell. In one embodiment, the cell comprises a vector comprising a nucleic acid sequence encoding a titinic protein.

In one embodiment, the cell is a prokaryotic cell or a eukaryotic cell. In another embodiment, the prokaryotic cell is a bacterial cell. In another embodiment, the eukaryotic cell is a primary cell or an immortalized cell or cell line. Other exemplary eukaryotic cells include CHO cells, HEK cells, neuronal cells (e.g., neurons or glia), and microglia cells. In certain embodiments, the host cell is stably transfected with a nucleic acid encoding a titinic protein. In any of the foregoing, the invention contemplates that a host cell expressing a titinic protein expresses the protein in the membrane, and further that the expressed protein retains one or more of the functions of native titinic protein. Eukaryotic cells can be from any species including, but not limited to, yeast, chick, fish, frog, mouse, rat, cat, dog, rabbit, cow, pig, horse, non-human primate, and human.

In one embodiment, the compound agonizes an activity (e.g., one or more function) of the titinic protein. In another embodiment, the compound antagonizes an activity (e.g., one or more function) of the titinic protein.

In one embodiment, the compound binds to the titinic protein.

In one embodiment of any of the foregoing, the compound can be any of a nucleic acid, a protein, or a small molecule. Exemplary nucleic acids include, but are not limited to, RNAi constructs, antisense oligonucleotides, and ribozymes. Exemplary proteins include, but are not limited to, antibodies. Exemplary small molecules have a molecular weight of less than approximately 600 daltons.

Candidate compounds according to any of the foregoing embodiments of this aspect of the invention, can be screened individually, in pools of more than one compounds, or by screening libraries of compounds. Furthermore, candidate compounds can be screened in single cells or in a culture of cells comprising more than one cell. Screening can optionally be by a high-throughput format.

In an eighth aspect, the invention provides a method of screening for titinic modulators. The method comprises combining a candidate bioactive agent with a cell expressing a titinic protein and detecting a change in enzymatic activity (phosphatase or kinase activity) in the cell in the presence of the bioactive agent in comparison to the absence of the bioactive agent. A candidate bioactive agent that modulates (increase or decrease) enzymatic activity in the cell is a titinic modulator.

In one embodiment, the cell expresses a titinic protein comprising four transmembrane domains and encoded by a nucleic acid that hybridizes under stringent conditions, including a wash step of 0.2×SSC at 65° C., to a nucleic acid sequence that is complementary to the sequence set forth in SEQ ID NO: 1. In another embodiment, the cell expresses a titinic protein according to any of the foregoing aspects or embodiments of the invention. The cell may express the titinic protein endogenously or titinic protein may be supplied or supplemented by exogenously expressing the protein in the cell. In one embodiment, the cell comprises a vector comprising a nucleic acid sequence encoding a titinic protein.

In one embodiment, the cell is a prokaryotic cell or a eukaryotic cell. In another embodiment, the prokaryotic cell is a bacterial cell. In another embodiment, the eukaryotic cell is a primary cell or an immortalized cell or cell line. Other exemplary eukaryotic cells include CHO cells, HEK cells, neuronal cells (e.g., neurons or glia), and microglia cells. In certain embodiments, the host cell is stably transfected with a nucleic acid encoding a titinic protein. In any of the foregoing, the invention contemplates that a host cell expressing a titinic protein expresses the protein in the membrane, and further that the expressed protein retains one or more of the functions of native titinic protein. Eukaryotic cells can be from any species including, but not limited to, yeast, chick, fish, frog, mouse, rat, cat, dog, rabbit, cow, pig, horse, non-human primate, and human.

In one embodiment, the compound agonizes an activity (e.g., one or more function) of the titinic protein. In another embodiment, the compound antagonizes an activity (e.g., one or more function) of the titinic protein.

In one embodiment, the compound binds to the titinic protein.

In one embodiment, the compound promotes an increase in enzymatic activity. In another embodiment, the compound promotes a decrease in enzymatic activity.

In one embodiment of any of the foregoing, the compound can be any of a nucleic acid, a protein, or a small molecule. Exemplary nucleic acids include, but are not limited to, RNAi constructs, antisense oligonucleotides, and ribozymes. Exemplary proteins include, but are not limited to, antibodies. Exemplary small molecules have a molecular weight of less than approximately 600 daltons.

Candidate compounds according to any of the foregoing embodiments of this aspect of the invention, can be screened individually, in pools of more than one compounds, or by screening libraries of compounds. Furthermore, candidate compounds can be screened in single cells or in a culture of cells comprising more than one cell. Screening can optionally be by a high-throughput format.

In a ninth aspect, the invention provides screening methods based on U.S. application Ser. No. 11,078,188, filed Mar. 11, 2005, the contents of which are hereby incorporated by reference in their entirety.

In a tenth aspect, the invention provides that any of the foregoing screening methods can be used to screen for candidate therapeutic agents to be used to prevent or treat inflammatory disease, Alzheimer's disease, or any condition associated with mutation in or misregulation of a titinic protein. Accordingly, the invention provides methods for screening for compounds that can be used to treat or prevent particular diseases or conditions in patients in need of treatment.

In one embodiment of any of the foregoing aspects and embodiments of the invention, the titinic protein is a voltage sensitive protein comprising four transmembrane domains, and the protein has one or more of the following functions: mediates membrane potential, mediates membrane voltage, and/or modulates enzymatic activity in a cell.

### DETAILED DESCRIPTION OF THE DRAWINGS

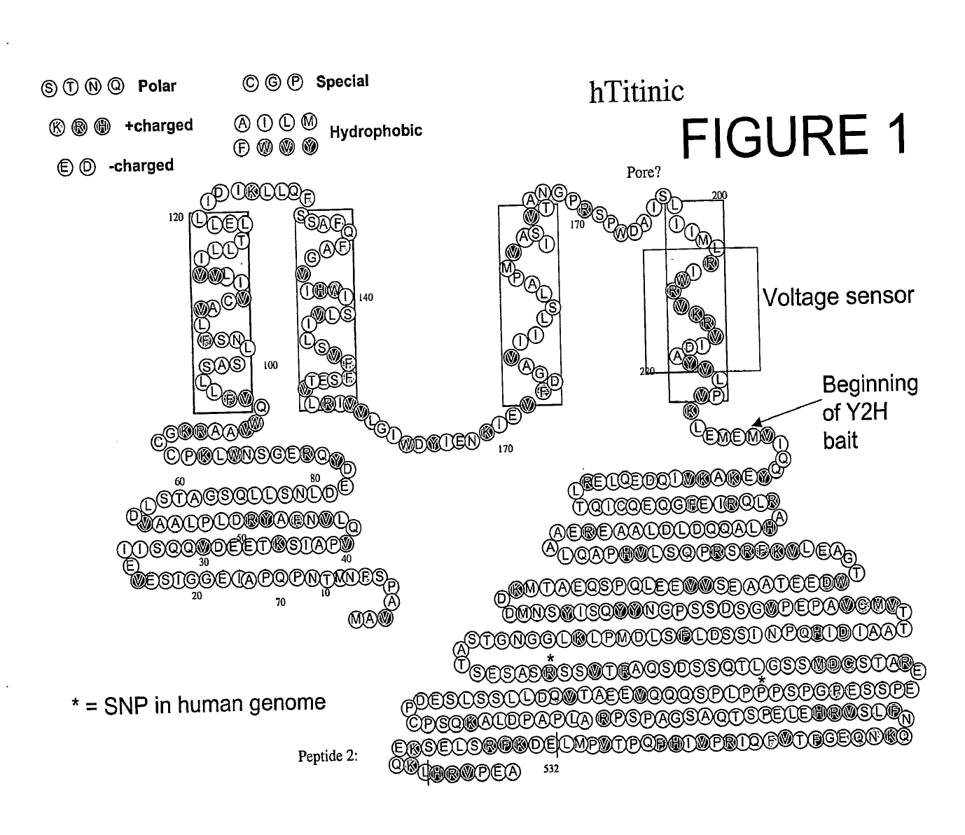

FIG. 1 provides a graphical depiction of the predicted structure of titinic protein. The protein depicted in FIG. 1 comprises an amino acid sequence represented in SEQ ID NO: 2. The location of single nucleotide polymorphisms found in the C-terminus is indicated by an asterisk. Also shown are the peptide sequence to which the polyclonal antibody used herein was raised against and the yeast two hybrid bait.

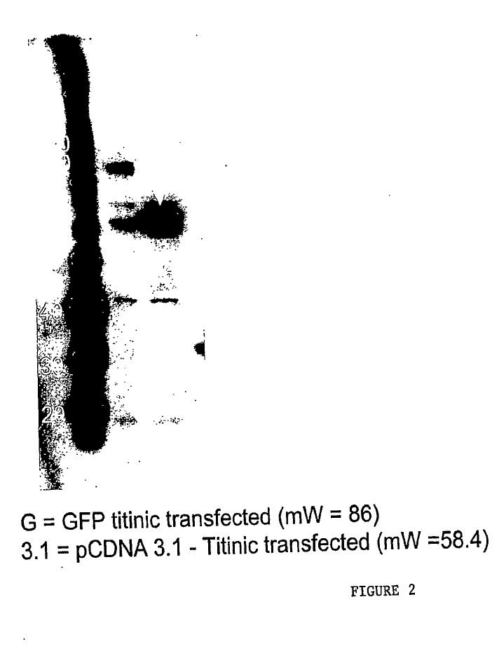

FIG. 2 is an exposure of a Western blot showing the expression of titinic in 293 cells transfected with a GFP-titinic vector (G) (MW=86 kDA) and a titinic vector (pcDNA3) (3.1) (MW=58.4 kDa). A Rabbit polyclonal antibody specific for CEDKFRSLESKEQKL was used (1:500).

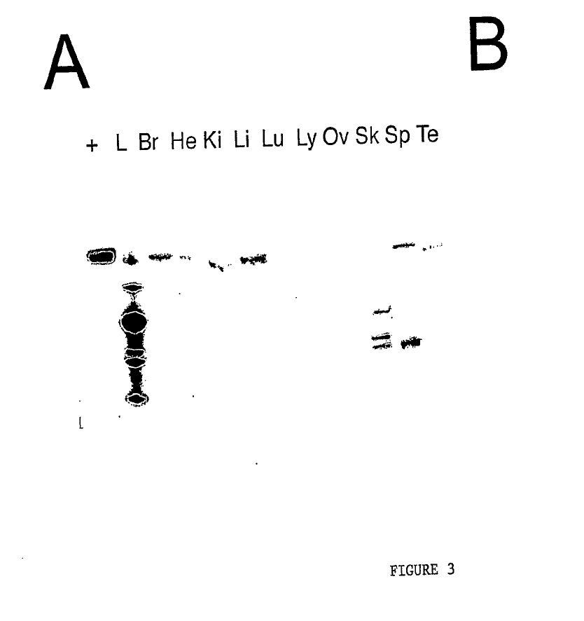

FIG. 3A is an exposure of a Western blot showing titinic expression in cells isolated ftom the brain (Br), heart (He), kidney (Ki), liver (Li), lung (Lu), lymph nodes (Ly), ovaries (Ov), skin (Sk), spleen (Sp), and testes (Te).

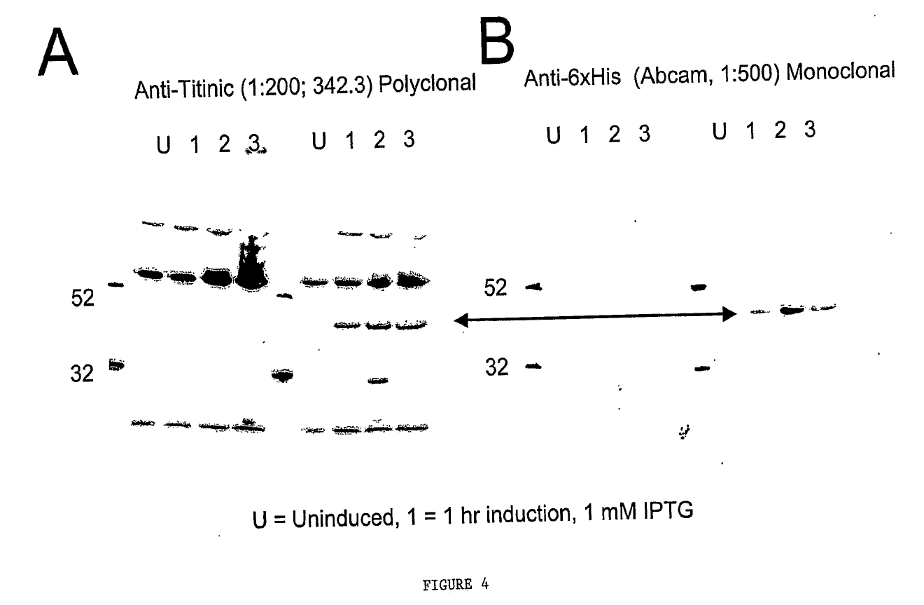

FIG. 3B is a photograph of the immunoblot described in FIG. 4A, which has been stained with Ponceau-S (0.2% ponceau-S in 0.5% acetic acid) to show protein loading levels to confirm even loading of the wells. Highest levels of protein were found in brain followed by kidney and there was virtually no expression detected in lung, lymph node and ovary.

FIGS. 4A and 4B are exposures of Western blots immunoblotted with a α-titinic and α-his antibody. Titinic protein levels were determined in BL21(DE3)pLysS cells transfected with a vector encoding an inducible His-tagged titinic protein in the absence of IPTG and 1, 2, and 3 hours following the addition of IPTG.

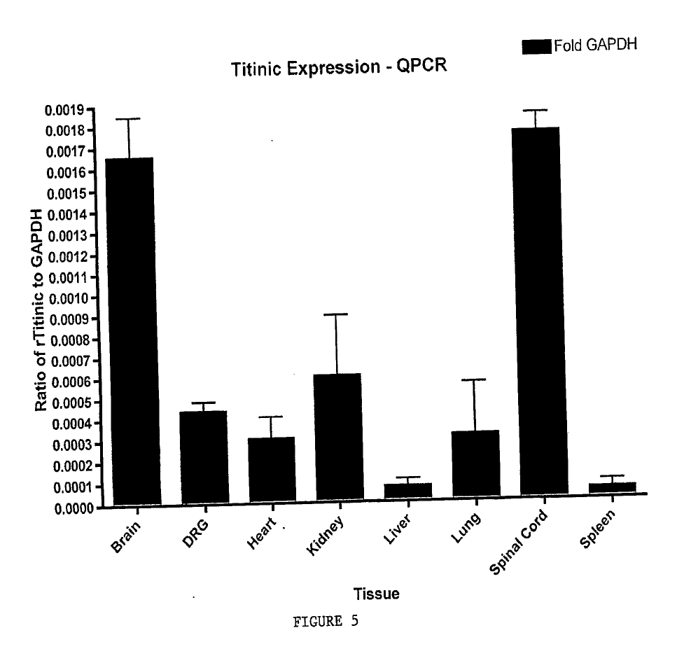

FIG. 5 is a bar graph showing the ratio of rat titinic transcripts relative to GAPDH transcripts in various tissues, as determined by real-time semi-quantitative PCR.

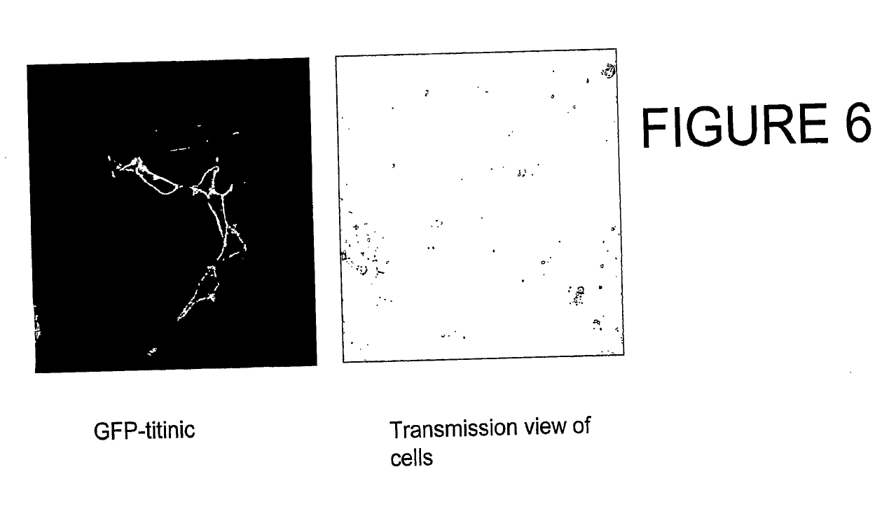

FIG. 6 is a series of confocal micrographs showing cell surface localization of the titinic protein in cells transfected with a vector that encodes a titinic protein operably linked to a green fluorescent protein (GFP).

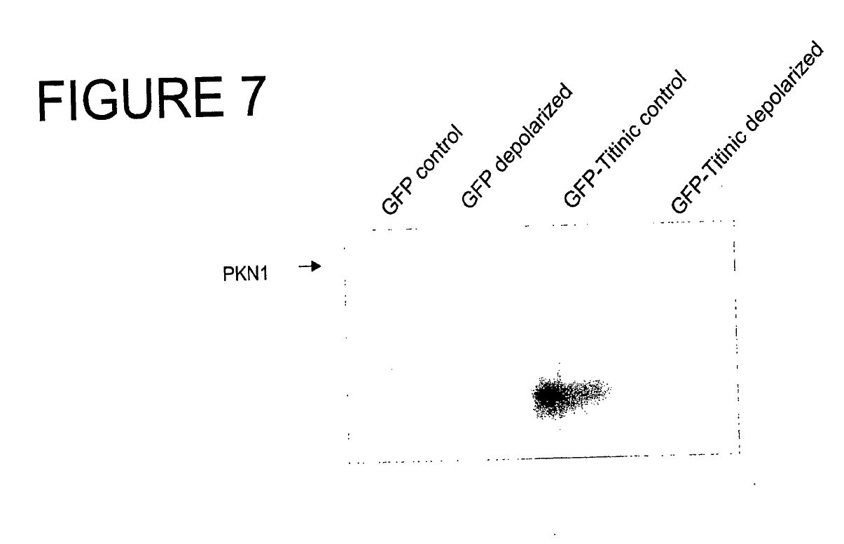

FIG. 7 is a photograph of an immunoblot representing a co-immunoprecipitation experiment to detect the in vivo interaction between titinic and PKN1.

### DETAILED DESCRIPTION OF THE INVENTION

The present invention provides methods and compositions relating to the newly identified titinic polypeptide. Titinic, a polypeptide of 531 amino acids, is encoded by a gene of 1596 nucleotides located on chromosome 15. This protein is predicted to have four transmembrane helices and an alpha coil domain, with the fourth putative transmembrane helix having a section of three arginine residues, similar to the voltage sensing domain in many voltage-gated ion channel. The C-terminal of the polypeptide is longer than the C-termini of most ion channels, indicating a potential functional role for this domain (see FIG. 1). This protein is expressed in various tissues including, for example, the brain, spine, dorsal root ganglia, and kidney (see FIGS. 4A and 6). It is localized at the plasma membrane (FIG. 6) where it is bound to PKN1 (activated by rho GTPase and phospholipids), and upon depolarization of the cell, titinic releases PKN1 (FIG. 7), thereby indicating a role for titinic in central and peripheral nervous system conditions involving hyperexcitability of neurons. The present invention provides a novel voltage sensitive protein (titinic) and several methods for screening to identify compounds that modulate one or more activities of this voltage sensitive protein, has significant utility.

### (i) Definitions

The articles “a” and “an” are used herein to refer to one or to more than one (i.e., to at least one) of the grammatical object of the article. By way of example, “an element” means one element or more than one element.

The terms “antagonist” and “inhibitor” are used interchangeably to refer to a compound that decreases or suppresses a biological activity or function, such as to repress an activity of a titinic voltage sensitive protein.

The term “agonist” is used to refer to a compound that increases or promotes a biological activity or function, such as to promote an activity of a titinic voltage sensitive protein.

An “effective amount” of, e.g., a titinic agonist or antagonist is an amount necessary to modulate a function of a titinic voltage sensitive protein. Without being bound by theory, an effective amount of a titinic agonist or antagonist for use in the methods of the present invention, includes an amount effective to modulate (increase or decrease) one or more in vitro or in vivo function of a titinic voltage sensitive protein. Exemplary functions include, but are not limited to, mediating intracellular pH, mediating hydrogen ion flux, mediating enzyme activity (e.g., kinase or phosphatase such as lipid phosphatase) mediating membrane potential, and mediating membrane voltage. Other exemplary functions include, but are not limited to, altering the function of one or more other ion channels in a cell to, for example, alter movement of other ions into or out of the cell in response to the cellular changes in hydrogen ion flux mediated by titinic.

The terms “compound” and “agent” are used interchangeably to refer to the candidate agonists and antagonists of the invention. In certain embodiments, the compounds are small organic or inorganic molecules, e.g., with molecular weights less than 7500 amu, preferably less than 5000 amu, and even more preferably less than 2000, 1500, 1000, or 500 amu. One class of small organic or inorganic molecules are non-peptidyl, e.g., containing 2, 1, or no peptide and/or saccharide linkages. In certain other embodiments, the compounds are peptidyl agents such as polypeptides or antibodies. In certain other embodiments, the compounds are nucleic acid agents such as sense or antisense oligonucleotides, RNAi constructs, ribozymes, and the like.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wildtype polynucleotide sequence or any change in a wildtype protein sequence. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent).

As used herein, the term “nucleic acid” refers to polynucleotides such as deoxyribonucleic acid (DNA), and, where appropriate, ribonucleic acid (RNA). The term should also be understood to include, as equivalents, analogs of RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

A “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

Variants may be full length or other than full length, and are within the scope of the present invention. Variants of the nucleic acids or proteins of the invention include, but are not limited to, molecules comprising regions that are substantially identical to the nucleic acids or proteins of the invention. In various embodiments, the variants are at least about 70%, 75%, 80%, 85%, 90%, 95%, 96%, 97%, 98%, 99%, or greater than 99% identical to a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions (Ausubel et al., 1987). Variants for use in the methods and compositions of the present invention retain one or more of the biological activities of the native sequence (e.g., of SEQ ID NO: 1 or 2).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

### (ii) Exemplary Compositions and Methods

The present invention provides a variety of titinic nucleic acid protein compositions. Titinic nucleic acid, proteins, bioactive fragments thereof, and compositions comprising any of the foregoing retain one or more of the following biological functions of titinic: mediates binding to signaling molecules such as PKN1 in a voltage dependent fashion, mediates cellular surface localization of PKN1 (e.g., to or away from the cellular surface), mediates intracellular pH, mediates hydrogen ion flux, mediates membrane potential, mediates membrane voltage, modulates superoxide production, and/or modulates phosphatase activity in a cell.

Polypeptides and peptide fragments: The present invention provides isolated, synthetically produced, and recombinantly produced titinic proteins. The invention further provides compositions and pharmaceutical compositions comprising titinic proteins. As outlined in detail herein, exemplary titinic proteins include proteins comprising an amino acid sequence at least 70%, 75%, 80%, 85%, 90%, 95%, 98%, or even 100% identical to SEQ ID NO: 2. Such proteins comprise 4 transmembrane domains and modulate proton transport. Further exemplary titinic proteins comprise a bioactive fragment of SEQ ID NO: 2 (or a variant thereof) that retains one or more of the functions of full length titinic protein.

Exemplary proteins or bioactive fragments according to the present invention retain one or more of the functions of a titinic protein as described herein.

Below we further describe various polypeptides according to the present invention. These polypeptides can be used, for example, to screen for compounds that agonize or antagonize a function of a titinic protein. Agonists or antagonist can be used, for example, in methods of modulating titinic function in vitro or in vivo. Such agents may be used in the development of pharmaceutical agents appropriate for administration to patients in need thereof.

In certain embodiments, the invention provides titinic proteins or compositions comprising titinic proteins. Titinic proteins and bioactive fragments thereof comprise an amino acid sequence at least 70%, 75%, 80%, 85%, 90%, 95%, 96%, 97%, 98%, 99%, or 100% identical to SEQ ID NO: 2. Furthermore, the invention contemplates titinic proteins that differ from SEQ ID NO: 2, at from one-ten positions (e.g., one, two, three, four, five, six, seven, eight, nine, or ten positions). Titinic proteins, variants, and bioactive fragments retain one or more of the functions of full length titinic.

Proteins according to the present invention also includes titinic proteins encoded by a nucleic acid sequence comprising a nucleotide sequence that hybridizes under stringent conditions, including a wash step of 0.2×SSC at 65° C., to a sequence that is complementary to the sequence represented in SEQ ID NO: 1. In certain embodiments, titinic proteins according to the invention are encoded by a nucleic acid sequence comprising a nucleotide sequence at least 70%, 75%, 80%, 85%, 90%, 95%, 97%, 98%, 99%, or 100% identical to SEQ ID NO: 1.

In any of the foregoing, the invention contemplates bioactive fragments of any of the foregoing titinic proteins or variant titinic proteins. Exemplary bioactive fragments include fragments of at least 200, 250, 300, 325, 350, or greater than 350 amino acid residues of a full length titinic protein (e.g., a protein comprising an amino acid sequence represented in SEQ ID NO: 2). Bioactive fragments for use in the methods of the present invention are capable of being expressed in a cellular membrane and retaining one or more of the biological activities of full length titinic protein. In any of the foregoing, titinic proteins for use in the methods of the present invention comprise four transmembrane domains and exhibit one or more functions associated with the native titinic protein, as described herein.

In addition to the polypeptides and fragments described in detail above, the present invention also pertains to isolated nucleic acids comprising nucleotide sequences that encode said polypeptides and fragments. The term nucleic acid as used herein is intended to include fragments as equivalents, wherein such fragments have substantially the same function as the full length nucleic acid sequence from which it is derived. Equivalent nucleotide sequences will include sequences that differ by one or more nucleotide substitutions, additions or deletions, such as allelic variants; and will, therefore, include sequences that differ from the nucleotide sequence represented in SEQ ID NO: 1. Equivalent sequences include those that vary from a known wildtype or variant sequence due to the degeneracy of the genetic code. Equivalent sequences may also include nucleotide sequences that hybridize under stringent conditions (i.e., equivalent to about 20-27° C. below the melting temperature (Tₘ) of the DNA duplex formed in about IM salt) to the nucleotide sequence of titinic polypeptide. Further examples of stringent hybridization conditions include a wash step of 0.2×SSC at 65° C. For the foregoing examples of equivalents to the titinic proteins of the present invention, one of skill in the art will recognize that an equivalent sequence encodes a polypeptide that retains one or more of the biological activities of native titinic. Specifically, the polypeptide retains one or more of the following biological activities of a full length, native titinic protein as described herein.

In one example, the invention contemplates a titinic protein or bioactive fragment thereof encoded or encodable by a nucleic acid sequence which hybridizes under stringent conditions, including a wash step of 0.2×SSC at 65° C., to a nucleic acid sequence that is complementary to the sequence represented in SEQ ID NO: 1 Equivalent nucleotide sequences for use in the methods described herein also include sequences which are at least 60% identical to a give nucleotide sequence. In another embodiment, the nucleotide sequence is at least 70%, 75%, 80%, 85%, 90%, 95%, 96%, 97%, 98%, 99%, or 100% identical to the nucleotide sequence of a titinic protein represented in SEQ ID NO: 1.

Nucleic acids having a sequence that differs from nucleotide sequences which encode a titinic protein due to degeneracy in the genetic code are also within the scope of the invention. Such nucleic acids encode functionally equivalent peptides but differ in sequence from wildtype sequences known in the art due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist. One skilled in the art will appreciate that these variations in one or more nucleotides (up to about 3-5% of the nucleotides) of the nucleic acids encoding polypeptides having one or more of the biological activities of a native titinic protein may exist among individuals of a given species due to natural allelic variation.

### (iii) Exemplary Expression Methods

The systems and methods described herein also provide expression vectors containing a nucleic acid encoding a titinic protein operably linked to at least one transcriptional regulatory sequence.

Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences may be used in these vectors to express nucleic acid sequences encoding the agents of this invention. Such useful expression control sequences, include, for example, a viral LTR, such as the LTR of the Moloney murine leukemia virus, the LTR of the Herpes Simplex virus-1, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs can be used to deliver nucleic acids encoding the subject polypeptides. Thus, another aspect of the invention features expression vectors for in vivo or in vitro transfection, viral infection and expression of a subject polypeptide in particular cell types. In one embodiment, such recombinantly produced polypeptides can be modified using standard techniques described herein, as well as other methodologies well known to one of skill in the art.

Expression constructs of the subject agents may be administered in biologically effective carriers, e.g. any formulation or composition capable of effectively delivering the recombinant gene to cells in vivo or in vitro. Approaches include insertion of the subject gene in viral vectors including recombinant retroviruses, adenovirus, adeno-associated virus, herpes simplex virus-1, lentivirus, mammalian baculovirus or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct, electroporation or CaPO₄ precipitation. One of skill in the art can readily select from available vectors and methods of delivery in order to optimize expression in a particular cell type or under particular conditions.

Retrovirus vectors and adeno-associated virus vectors have been frequently used for the transfer of exogenous genes. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes. Thus, recombinant retrovirus can be constructed in which part of the retroviral coding sequence (gag, pol env) has been replaced by nucleic acid encoding one of the subject proteins rendering the retrovirus replication defective. The replication defective retrovirus is then packaged into virions through the use of a helper virus by standard techniques which can be used to infect a target cell. Protocols for producing recombinant retroviruses and for infecting cells in vitro or in vivo with such viruses can be found in Current Protocols in Molecular Biology, Ausubel, F. M. et al. (eds.) Greene Publishing Associates, (2000), and other standard laboratory manuals. Examples of suitable retroviruses include pBPSTR1, pLJ, pZIP, pWE and pEM which are known to those skilled in the art. Examples of suitable packaging virus lines for preparing both ecotropic and amphotropic retroviral systems include ψCrip, ψCre, ψ2, ψAm, and PA317.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein; or coupling cell surface receptor ligands to the viral env proteins. Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences which control expression of the gene of the retroviral vector such as tetracycline repression or activation.

Another viral gene delivery system which has been employed utilizes adenovirus-derived vectors. The genome of an adenovirus can be manipulated so that it encodes and expresses a gene product of interest but is inactivated in terms of its ability to replicate in a normal lytic viral life cycle. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they can be used to infect a wide variety of cell types, including airway epithelium, endothelial cells, hepatocytes, and muscle cells. Furthermore, the virus particle is relatively stable and amenable to purification and concentration, and as above, can be modified so as to affect the spectrum of infectivity.

Yet another viral vector system is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158: 97-129). It is also one of the few viruses that may integrate its DNA into non-dividing cells, and exhibits a high frequency of stable integration.

Another viral delivery system is based on herpes simplex-1 (HSV-1). HSV-1 based vectors have been shown to infect a variety of cells including post mitotic cells such as neuronal cells (Agudo et al. (2002) Human Gene Therapy 13: 665-674; Latchman (2001) Neuroscientist 7: 528-537; Goss et al. (2002) Diabetes 51: 2227-2232; Glorioso (2002) Current Opin Drug Discov Devel 5: 289-295; Evans (2002) Clin Infect Dis 35: 597-605; Whitley (2002) Journal of Clinical Invest 110: 145-151; Lilley (2001) Curr Gene Ther 1: 339-359).

The above cited examples of viral vectors are by no means exhaustive. However, they are provided to indicate that one of skill in the art may select from well known viral vectors, and select a suitable vector for expressing a particular protein in a particular cell type.

In addition to viral transfer methods, such as those illustrated above, non-viral methods can be used to express a subject polypeptide. Many nonviral methods of gene transfer rely on normal mechanisms used by cells for the uptake and intracellular transport of macromolecules. Exemplary gene delivery systems of this type include liposomal derived systems, poly-lysine conjugates, and artificial viral envelopes.

It may sometimes be desirable to introduce a nucleic acid directly to a cell, for example a cell in culture or a cell in an animal. Such administration can be done by injection of the nucleic acid (e.g., DNA, RNA) directly at the desired site. Such methods are commonly used in the vaccine field, specifically for administration of “DNA vaccines”, and include condensed DNA (U.S. Pat. No. 6,281,005).

In addition to administration of nucleic acids, the systems and methods described herein contemplate that polypeptides may be administered directly. Some proteins, for example factors that act extracellularly by contacting a cell surface receptor, such as growth factors, may be administered by simply contacting cells with said protein. For example, cells are typically cultured in media which is supplemented by a number of proteins such as FGF, TGFβ, insulin, etc. These proteins influence cells by simply contacting the cells.

In another embodiment, a polypeptide is directly introduced into a cell. Methods of directly introducing a polypeptide into a cell include, but are not limited to, protein transduction and protein therapy. For example, a protein transduction domain (PTD) can be fused to a nucleic acid encoding a particular agent, and the fusion protein is expressed and purified. Fusion proteins containing the PTD are permeable to the cell membrane, and thus cells can be directly contacted with a fusion protein (Derossi et al. (1994) Journal of Biological Chemistry 269: 10444-10450; Han et al. (2000) Molecules and Cells 6: 728-732; Hall et al. (1996) Current Biology 6: 580-587; Theodore et al. (1995) Journal of Neuroscience 15: 7158-7167).

Although some protein transduction based methods rely on fusion of a polypeptide of interest to a sequence which mediates introduction of the protein into a cell, other protein transduction methods do not require covalent linkage of a protein of interest to a transduction domain. At least two commercially available reagents exist that mediate protein transduction without covalent modification of the protein (Chariot™, produced by Active Motif, www.activemotif.com and Bioporter® Protein Delivery Reagent, produced by Gene Therapy Systems, www.genetherapysystems.com).

Briefly, these protein transduction reagents can be used to deliver proteins, peptides and antibodies directly to cells including mammalian cells. Delivery of proteins directly to cells has a number of advantages. Firstly, many current techniques of gene delivery are based on delivery of a nucleic acid sequence which must be transcribed and/or translated by a cell before expression of the protein is achieved. This results in a time lag between delivery of the nucleic acid and expression of the protein. Direct delivery of a protein decreases this delay. Secondly, delivery of a protein often results in transient expression of the protein in a cell.

As outlined herein, protein transduction mediated by covalent attachment of a PTD to a protein can be used to deliver a protein to a cell. These methods require that individual proteins be covalently appended with PTD moieties. In contrast, methods such as Chariot™ and Bioporter® facilitate transduction by forming a noncovalent interaction between the reagent and the protein. Without being bound by theory, these reagents are thought to facilitate transit of the cell membrane, and following internalization into a cell the reagent and protein complex disassociates so that the protein is free to function in the cell.

This application also describes methods for producing the subject polypeptides. For example, a host cell transfected with a nucleic acid vector directing expression of a nucleotide sequence encoding the subject polypeptides can be cultured under appropriate conditions to allow expression of the peptide to occur. The polypeptide may be secreted and isolated from a mixture of cells and medium containing the recombinant polypeptide. Alternatively, the peptide may be expressed cytoplasmically and the cells harvested, lysed and the protein isolated. A cell culture includes host cells, media and other by-products. Suitable media for cell culture are well known in the art. The recombinant polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In one example, the recombinant polypeptide is a fusion protein containing a domain which facilitates its purification, such as a GST fusion protein. In another example, the subject recombinant polypeptide may include one or more additional domains which facilitate immunodetection, purification, and the like. Exemplary domains include HA, FLAG, GST, His, and the like. Further exemplary domains include a protein transduction domain (PTD) which facilitates the uptake of proteins by cells. Recombinantly expressed proteins can be modified using methods disclosed herein, as well as those well known to one of skill in the art.

This application also describes a host cell which expresses a recombinant form of the subject polypeptides. The host cell may be a prokaryotic or eukaryotic cell. Thus, a nucleotide sequence derived from the cloning of a protein encoding all or a selected portion (either an antagonistic portion or a bioactive fragment) of the full-length protein, can be used to produce a recombinant form of a polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known proteins, e.g. insulin, interferons, human growth hormone, IL-1, IL-2, and the like. Similar procedures, or modifications thereof, can be employed to prepare recombinant polypeptides by microbial means or tissue-culture technology in accord with the subject invention. Such methods are used to produce experimentally useful proteins that include all or a portion of the subject nucleic acids. For example, such methods are used to produce fusion proteins including domains which facilitate purification or immunodetection, and to produce recombinant mutant forms of a protein).

The recombinant genes can be produced by ligating a nucleic acid encoding a protein, or a portion thereof, into a vector suitable for expression in either prokaryotic cells, eukaryotic cells, or both. Expression vectors for production of recombinant forms of the subject polypeptides include plasmids and other vectors. For instance, suitable vectors for the expression of a polypeptide include plasmids of the types: pBR322-derived plasmids, pEMBL-derived plasmids, pEX-derived plasmids, pGEX-derived plasmids, pTrc-His-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae.

Many mammalian expression vectors contain both prokaryotic sequences, to facilitate the propagation of the vector in bacteria, and one or more eukaryotic transcription units that are expressed in eukaryotic cells. The pcDNA3.1, pcDNA5, Qbi25FC3, pcDNAI/amp, pcDNAI/neo, pRc/CMV, pSV2gpt, pSV2neo, pSV2-dhfr, pTk2, pRSVneo, pMSG, pSVT7, pko-neo, pBacMam-2, and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. For other suitable expression systems for both prokaryotic and eukaryotic cells, as well as general recombinant procedures, see Molecular Cloning A Laboratory Manual, 3rd Ed., ed. by Sambrook and Russell (Cold Spring Harbor Laboratory Press: 2001).

In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the 13-gal containing pBlueBac III).

The present invention also makes available isolated polypeptides which are isolated from, or otherwise substantially free of other cellular and extracellular proteins. The term “substantially free of other cellular or extracellular proteins” (also referred to herein as “contaminating proteins”) or “substantially pure or purified preparations” are defined as encompassing preparations having less than 20% (by dry weight) contaminating protein, and preferably having less than 5% contaminating protein. Functional forms of the subject proteins can be prepared as purified preparations by using a cloned gene as described herein. By “purified”, it is meant, when referring to peptide or nucleic acid sequences, that the indicated molecule is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water and buffers can be present). The term “pure” as used herein preferably has the same numerical limits as “purified” immediately above.

“Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

Isolated peptidyl portions of proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. Chemically synthesized proteins can be modified using methods described herein, as well as methods well known in the art.

The recombinant polypeptides of the present invention also include versions of those proteins that are resistant to proteolytic cleavage. Variants of the present invention also include proteins which have been post-translationally modified in a manner different than the authentic protein. Modification of the structure of the subject polypeptides can be for such purposes as enhancing therapeutic or prophylactic efficacy, or stability (e.g., ex vivo shelf life and resistance to proteolytic degradation in vivo). Such modified peptides, when designed to retain at least one activity of the naturally-occurring form of the protein, are considered functional equivalents of the polypeptides described in more detail herein. Such modified peptides can be produced, for instance, by amino acid substitution, deletion, or addition.

For example, it is reasonable to expect that, in some instances, an isolated replacement of a leucine with an isoleucine or valine, an aspartate with a glutamate, a threonine with a serine, or a similar replacement of an amino acid with a structurally related amino acid (e.g., isosteric and/or isoelectric mutations) may not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 5th ed. by Berg, Tymoczko and Stryer, WH Freeman and Co.: 2002). Whether a change in the amino acid sequence of a peptide results in a functional variant (e.g. functional in the sense that it acts to mimic or antagonize the wild-type form) can be determined by assessing the ability of the variant peptide to produce a response in cells in a fashion similar to the wild-type protein, or competitively inhibit such a response. Polypeptides in which more than one replacement has taken place can readily be tested in the same manner.

Advances in the fields of combinatorial chemistry and combinatorial mutagenesis have facilitated the making of polypeptide variants (Wissmanm et al. (1991) Genetics 128: 225-232; Graham et al. (1993) Biochemistry 32: 6250-6258; York et al. (1991) Journal of Biological Chemistry 266: 8495-8500; Reidhaar-Olson et al. (1988) Science 241: 53-57). Given one or more assays for testing polypeptide variants, one can assess whether a given variant retains one or more of the biological activities of the corresponding native polypeptide.

### (iv) Further Compositions and Cell-Based Expression

In another aspect, the present invention provides compositions and pharmaceutical compositions comprising, consisting of, or consisting essentially of particular titinic polypeptides or nucleic acids. Such polypeptides and nucleic acids can be used, for example, in drug screening assays or to make primers or probes to study the expression or activity of titinic in cells, tissues, or organisms. As used herein, the term “isolated” when used to refer to nucleic acid and polypeptide compositions refers to nucleic acids or polypeptides existing in a state other than the state in which they exist in nature. In other words, the term is used to denote some level of separation from other proteins and cellular components with which the protein is endogenously found. Isolated, when used in this context, does not necessarily mean that the protein or nucleic acid is provided in a purified form. Additionally, the term “isolated” is not intended to imply that the polypeptide or nucleic acid is isolated from an organism. Rather, the term also includes recombinantly produced nucleic acids and polypeptides.

In certain embodiments, the invention provides an isolated polypeptide comprising, consisting of, or consisting essentially of an amino acid sequence 80%, 85%, 90%, 95%, 97%, 98%, 99%, or 100% identical to the amino acid sequence represented in SEQ ID NO: 2. Such polypeptides may include the identical sequence, or may include one, two, or three conservative substitutions, additions, or deletions. In certain other embodiments, the invention provides an isolated polypeptide encoded by a nucleic acid sequence comprising, consisting of, or consisting essentially of a nucleotide sequence represented in SEQ ID NO: 1, or by a nucleotide sequence that varies from SEQ ID NO: 1 due to the degeneracy of the genetic code, or by a nucleotide sequence that hybridizes under stringent conditions, including a wash step of 0.2×SSC at 65° C., to a sequence represented in SEQ ID NO: 1.

In certain other embodiments, the invention provides an isolated nucleic acid comprising, consisting of, or consisting essentially of a nucleotide sequence represented in SEQ ID NO; 1, or by a nucleotide sequence that varies from SEQ ID NO: 1 due to the degeneracy of the genetic code, or by a nucleotide sequence that hybridizes under stringent conditions, including a wash step of 0.2×SSC at 65° C., to a sequence represented in SEQ ID NO: 1. In other embodiments, the invention provides an isolated nucleic acid comprising, consisting of, or consisting essentially of a nucleotide sequence which encodes a polypeptide comprising an amino acid sequence represented in SEQ ID No. 2.

In other embodiments, the invention provides an expression vector, which replicates in at least one of a prokaryotic cell and eukaryotic cell. The expression vector comprises any of the foregoing titinic nucleic acids. Similarly provided are cells comprising these expression vectors, which cells express the titinic protein encoded by the expressed nucleic acid. For example, these cells express titinic protein in the membrane. In certain embodiments, the expressed polypeptide retain one or more functions of titinic. Additionally provided are methods of producing a polypeptide. The method includes culturing one of the foregoing cells (e.g., a cell expressing titinic polypeptide) in a suitable cell culture medium to express said polypeptide.

In certain embodiments, the cell is transiently transfected with the expression vector and transiently expresses titinic protein. In certain other embodiments, the cell is stably transfected with the expression vector and a stable cell line expressing titinic is established. In certain embodiments, the cell comprising the expression vector does not endogenously express titinic protein (e.g., the cell does not express appreciable levels of titinic protein in the absence of the expression vector). In other embodiments, the cell comprising the expression vector endogenously expresses titinic protein. In certain aspects of any of the foregoing, suitable cell-based expression systems comprise expressing titinic at the cell surface.

In certain embodiments, cells expressing titinic, for example, cells manipulated to comprise a titinic expression vector, can be used in screening assays to identify compounds that modulate a function of titinic. Suitable cells include, without limitation, prokaryotic cells and eukaryotic cells. Exemplary eukaryotes include vertebrates and invertebrates. Exemplary eukaryotes include, but are not limited to, humans, mice, rats, cats, dogs, rabbits, sheep, cows, horses, goats, non-human primates, frogs, toads, fish, chicken, flies, worms, and yeast. Exemplary prokaryotes include bacteria. When “a cell” is referred to, it is understood to refer to screening in at least one cell (e.g., a single cell or a culture of cells). Cells may be provided in suspension or grown adherently. Cells of any developmental time and tissue can be used. Exemplary cells include embryonic cells, larval cells, juvenile cells, fetal cells, and adult cells. Exemplary cells and cell line may be derived from any tissue or cell type. Cells include primary cells and transformed cell lines.

In certain embodiments, as noted above, the invention contemplates an expression vector which comprises a coding sequence for a titinic protein, as provided herein. A “vector” is a replicon, such as plasmid, phage or cosmid, to which another DNA segment may be attached. The term “vector” refers to a nucleic acid molecule capable of transporting another nucleic acid to which it has been linked. One type of vector is an episome which is a nucleic acid capable of extra-chromosomal replication. Vectors capable of autonomous replication and/or expression of nucleic acids to which they are linked may also be used. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops which, in their vector form are not bound to the chromosome. However, the invention is intended to include such other forms of expression vectors which serve equivalent functions and which become known in the art subsequently hereto.

A DNA or nucleic acid “coding sequence” is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence of the present invention can include, but is not limited to, cDNA from eukaryotic mRNA, genomic DNA sequences from eukaryotic (e.g., mammalian) DNA, and synthetic DNA sequences. A polyadenylation signal and transcription termination sequence may be located 3′ of the coding sequence.

Nucleic acid or DNA regulatory sequences or regulatory elements are transcriptional and translational control sequences, such as promoters, enhancers, polyadenylation signals, and terminators, that provide for and/or regulate expression of a coding sequence in a host cell. Regulatory sequences for directing expression of eukaryotic ion channels and detectable markers of certain embodiments are art-recognized and may be selected by a number of well understood criteria. Examples of regulatory sequences are described in Goeddel, Gene Expression Technology: Methods in Enzymology (Academic Press, San Diego, Calif. (1990)). For instance, any of a wide variety of expression control sequences that control the expression of a DNA sequence when operatively linked to it may be used in these vectors to express DNA sequences encoding the ion channels and detectable markers. Such useful expression control sequences, include, for example, the early and late promoters of SV40, beta2 tubulin, adenovirus or cytomegalovirus immediate early promoter, the lac system, the trp system, the TAC or TRC system, T7 promoter whose expression is directed by T7 RNA polymerase, the promoter for 3-phosphoglycerate kinase or other glycolytic enzymes, the promoters of acid phosphatase, e.g., PhoS, and the promoters of the yeast α-mating factors and other sequences known to control the expression of genes of prokaryotic or eukaryotic cells or their viruses, and various combinations thereof. It should be understood that the design of the expression vector may depend on such factors as the choice of the host cell to be transformed. Moreover, the vector's copy number, the ability to control that copy number and the expression of any other protein encoded by the vector, such as antibiotic markers, should also be considered.

The invention contemplates the use of any promoter that can drive the expression of a titinic protein in prokaryotic or eukaryotic cells. As used herein, the term “promoter” means a DNA sequence that regulates expression of a selected DNA sequence operably linked to the promoter, and which effects expression of the selected DNA sequence in cells. A “promoter” generally is a DNA regulatory element capable of binding RNA polymerase in a cell and initiating transcription of a coding sequence. For example, the promoter sequence may be bounded at its 3′ terminus by the transcription initiation site and extend upstream (5′ direction) to include the minimum number of bases or elements necessary to initiate transcription at levels detectable above background. Within the promoter sequence may be found a transcription initiation site, as well as protein binding domains responsible for the binding of RNA polymerase. Eukaryotic promoters will often, but not always, contain “TATA” boxes and “CAAT” boxes. Various promoters, including inducible promoters, may be used to drive the various vectors of the present invention.

The term “promoter” also encompasses prokaryotic and/or eukaryotic promoters and promoter elements. The term “promoter” as used herein encompasses “cell specific” promoters, i.e. promoters, which effect expression of the selected DNA sequence only in specific cells (e.g., cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e., expression levels can be controlled).

As detailed above and in certain embodiments, the invention contemplates expression vectors comprising a titinic nucleic acid sequence and capable of expressing titinic protein. When expressed in cells, these vectors express titinic protein, preferably functional protein.

Cells expressing a titinic expression vector may be assayed to confirm expression of titinic protein. For example, protein expression may be confirmed using Western blot analysis, immunocytochemistry, or immunohistochemistry. Additionally or alternatively, titinic function can be assessed using, for example, calcium imaging analysis to evaluate ion flux or electrophysiological methods (e.g., patch clamp analysis) to evaluate current.

### Screening Methods

The present invention provides a number of screening methods for identifying compounds that modulate titinic activity. Compounds identified as modulating an activity of a titinic protein include agonists that increase an activity and antagonists that decrease an activity. Compounds having the ability to modulate the activity of titinic are identified as being useful for treating central and peripheral nervous system conditions involving the hyperexcitability of neurons including, for example, epilepsy, pain, and cerebral ischemic disease.

For any of the numerous screening methods provided herein, one or more candidate compounds (or a library of candidate compounds) is contacted or administered to a cell expressing a titinic protein. The cell may be a cell that endogenously expresses titinic. Alternatively, regardless of whether the cell does or does not endogenously expressed titinic, the cell used in screening can be engineered to express an exogenously supplied titinic protein.

Cells (individual cells or cultures of cells) are contacted with the candidate compound or compounds. The method comprises detecting a change in a variable that can act as a read-out or proxy for one or more biological functions of titinic as described herein. For example, titinic activity may be measured by its ability to interact with and modulate the activity of PKN1. This variable is assessed in the presence versus the absence of the candidate compound. Compounds that modulate this variable are identified as compounds that modulate an activity of a titinic protein.

The invention contemplates methods of screening for candidate compounds by detecting changes in ion flux. The invention contemplates methods of screening for candidate compounds by detecting changes in production of superoxide ions. The invention contemplates methods of screening for candidate compounds by detecting changes in enzymatic activity (e.g., phosphatase or kinase activity) in the cell. The invention contemplates methods of screening for candidate compounds by detecting changes in membrane voltage or membrane potential.

For any of the screening methods described herein, the cell expresses (either endogenously and/or exogenously supplied) a titinic protein. In one embodiment, the cell expresses a titinic protein comprising four transmembrane domains and encoded by a nucleic acid that hybridizes under stringent conditions, including a wash step of 0.2×SSC at 65° C., to a nucleic acid sequence that is complementary to the sequence set forth in SEQ ID NO: 1. In another embodiment, the cell expresses a titinic protein according to any of the foregoing aspects or embodiments of the invention. The cell may express the titinic protein endogenously or titinic protein may be supplied or supplemented by exogenously expressing the protein in the cell.

In one embodiment, the cell comprises a vector comprising a nucleic acid sequence encoding a titinic protein.

Cells for use in the screening methods of the invention can be prokaryotic cells or eukaryotic cells. The screening methods can be done on individual cells, small numbers of cultures dishes of cells, or in a high-throughput format. In another embodiment, the prokaryotic cell is a bacterial cell. In another embodiment, the eukaryotic cell is a primary cell or an immortalized cell or cell line. Other exemplary eukaryotic cells include CHO cells, HEK cells, neuronal cells (e.g., neurons or glia), and microglia cells. In certain embodiments, the host cell is stably transfected with a nucleic acid encoding a titinic protein. In any of the foregoing, the invention contemplates that a host cell expressing a titinic protein expresses the protein in the membrane, and further that the expressed protein retains one or more of the functions of native titinic protein. Eukaryotic cells can be from any species include, but not limited to, yeast, chick, fish, frog, mouse, rat, cat, dog, rabbit, cow, pig, horse, non-human primate, and human.

Another screening assay contemplated by the present invention involves combining a candidate bioactive agent with a cell expressing a titinic protein and a pH sensitive receptor protein; and detecting the activity of the pH sensitive reporter protein in the presence versus the absence of the bioactive agent. A change in the activity of the pH sensitive protein in the presence versus the absence of the bioactive agent will be used to identify a compound as a titinic modulator.

Another screening assay contemplated by the present invention is described in U.S. application Ser. No. 11,078,188, filed Mar. 11, 2005, the contents of which are hereby incorporated by reference in their entirety. Titinic protein can be expressed in the prokaryotic cell system described in application Ser. No. 11,078,188, and this system can be used to screen for compounds that modulate an activity of the titinic protein.

The invention further provides a screening method that involves contacting a candidate compound with a PKN1 and titinic protein. If desired, the contacting event may occur inside a cell. Alternatively, such contacting may occur in cell-free conditions. A candidate compound that modulates the binding of PKN1 and titinic protein is identified as a compound having the ability to modulate PKN1 activity. Optionally, if such contacting occurs in a cell, the cell may be in a polarized or depolarized state. A compound having the ability to reduce the ability of titinic to bind PKN1 is identified as a compound useful for treating a central or peripheral nervous system condition involving hyperexcitability of neurons.

The invention provides that any of the foregoing screening methods can be used to screen for candidate therapeutic agents to be used to prevent or treat inflammatory disease, Alzheimer's disease, or any condition associated with mutation in or misregulation of a titinic protein.

Compounds identified as titinic modulators using the screening methods of the present invention can be further tested or used in one or more animal models. For example, identified compounds can be tested in an animal model of pain, epilepsy, cerebral ischemic disease, stroke, inflammation or Alzheimer's disease. Eventually such compounds would be administered to human patients in need thereof.

Compounds administered to human or non-human animals would be administered as pharmaceutical compositions formulated in a pharmaceutically acceptable carrier or excipient. One of skill in the art would formulate such pharmaceutical compositions in a manner appropriate for the compound, disease, patient, and route of administration. One of skill in the art would select an appropriate route of administration depending on the disease to be treated, the patient, and the biological and pharmacological properties of the compound.

### Compounds

The present invention contemplates compounds (used interchangeably with agents) that function as modulators of the activity of a membrane protein (e.g., a protein that mediates membrane flux), thereby modulating ion flux. By “agents” or “candidate agents” herein is meant to include nucleic acids, peptides, polypeptides, peptidomimetics, small organic molecules, inorganic molecules, antisense oligonucleotides, RNAi constructs, antibodies, and ribozymes that function as antagonistic or agonistic candidate agents.

A compound of the invention may comprise an agonist of a titinic protein or, alternatively, an antagonist of a titinic protein. In certain embodiments, a compound of the invention may function by binding to the titinic protein directly or indirectly.

The term “agonist,” as used herein, is meant to refer to an agent that mimics or upregulates (e.g., potentiates or supplements) bioactivity of the protein of interest. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide of interest with another molecule, e.g., a target molecule, a target peptide, or nucleic acid.

By “antagonist” herein is meant an agent that downregulates (e.g. suppresses or inhibits) bioactivity of the protein of interest. An antagonist can be a compound which inhibits or decreases the interaction between a protein and another molecule. For example, an antagonist of the invention may compete for binding to an ion channel against the ion channel's naturally occurring ligands or agonists. An antagonist can also be a compound that downregulates expression of the gene of interest or which reduces the amount of the wild type protein present.

Agents that stimulate the channel's activity are useful as agonists in disease states or conditions characterized by insufficient channel signaling (e.g., as a result of insufficient activity of the channel ligand). Agents that block ligand-mediated channel signaling are useful as the channel antagonists to treat disease states or conditions characterized by excessive channel signaling. In addition, the ion channel-modulating agents in general, as well as channel polynucleotides and polypeptides, are useful in diagnostic assays for such diseases or conditions.

The agents of the invention exhibit a variety of chemical structures, which can be generally grouped into non-peptide mimetics of natural ion channel ligands, peptide and non-peptide allosteric effectors of ion channels, and peptides that may function as activators or inhibitors (competitive, uncompetitive and non-competitive) (e.g., antibody products) of a membrane protein. The invention does not restrict the sources for suitable agents, which may be obtained from natural sources such as plant, animal or mineral extracts, or non-natural sources such as small molecule libraries, including the products of combinatorial chemical approaches to library construction, and peptide libraries.

Other assays can be used to examine enzymatic activity including, but not limited to, photometric, radiometric, HPLC, electrochemical, and the like, which are described in, for example, Enzyme Assays: A Practical Approach (R. Eisenthal and M. J. Danson, 1992, Oxford University Press).

Candidate agents contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides or organic molecules.

Chemical libraries consist of random chemical structures, some of which are analogs of known compounds or analogs of compounds that have been identified as “hits” or “leads” in other drug discovery screens, some of which are derived from natural products, and some of which arise from non-directed synthetic organic chemistry.

Natural product libraries are collections of microorganisms, animals, plants, or marine organisms that are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282: 63-68 (1998). Combinatorial libraries are composed of large numbers of peptides, oligonucleotides, or organic compounds as a mixture These libraries are relatively easy to prepare by traditional automated synthesis methods, PCR, cloning, or proprietary synthetic methods. Of particular interest are-non-peptide combinatorial libraries. Still other libraries of interest include peptide, protein, peptidomimetic, multiparallel synthetic collection, recombinatorial, polypeptide, antibody, and RNAi libraries. For a review of combinatorial chemistry and libraries created therefrom, see Myers, Curr. Opin. Biotechnol. 8: 701-707 (1997). Identification of modulators through use of the various libraries described herein permits modification of the candidate “hit” or “lead” to optimize the capacity of the “hit” to modulate activity.

The following are illustrative examples of compounds that can be screened using the methods of the present invention. Such compounds, if identified as compounds that modulate a function of a titinic channel, could be further analyzed in vitro or in vivo or formulated for in vivo delivery as a pharmaceutical composition.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein and therefore may generally comprise antagonistic agents of the present invention, if they target a eukaryotic ion channel. Alternatively, antisense molecules may comprise agonistic agents of the present invention, if they target an antagonistic mRNA or protein modulator of the eukaryotic ion channel of choice.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652; PCT Publication No. WO88/09810) or the blood-brain barrier (see, e.g., PCT Publication No. WO89/10134), hybridization-triggered cleavage agents (see, e.g., Krol et al., 1988, BioTechniques 6: 958-976) or intercalating agents (see, e.g., Zon, 1988, Pharm. Res. 5: 539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93: 14670 and in Eglom et al. (1993) Nature 365: 566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an anomeric oligonucleotide. An anomeric oligonucleotide forms specific double-stranded hybrids with complementary RNA in which, contrary to the usual units, the strands run parallel to each other (Gautier et al., 1987, Nucl. Acids Res. 15: 6625-6641). The oligonucleotide is a 2′-0-methylribonucleotide (Inoue et al., 1987, Nucl. Acids Res. 15: 6131-6148), or a chimeric RNA-DNA analogue (Inoue et al., 1987, FEBS Lett. 215: 327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16: 3209), methylphosphonate oligonucleotides can be prepared by use of controlled pore glass polymer supports (Sarin et al., 1988, Proc. Natl. Acad. Sci. U.S.A. 85: 7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein.

To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related protein such as isoforms or functionally redundant protein. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site or the cells, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in prokaryotic cells (e.g., in an expression system or screening assay of the invention) or eukaryotic cells (e.g., in a pharmaceutical preparation of the invention). Such promoters can be inducible or constitutive. Such promoters include but are not limited to: the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al, 1982, Nature 296: 39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g.; systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo. As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell (e.g., a prokaryotic cell in a screening assay of the invention, or a eukaryotic cell or mammalian cell in a pharmaceutical preparation of the invention). In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAI constructs contain a nucleotide sequence that hybridizes under physiological conditions of the cell to the nucleotide sequence of at least a portion of the mRNA transcript for the gene to be inhibited (i.e., the “target” gene), e.g., a eukaryotic ion channel or a protein modulator of the eukaryotic ion channel. The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Methods of chemically modifying RNA molecules can be adapted for modifying RNAi constructs (see, for example, Heidenreich et al. (1997) Nucl. Acids Res, 25: 776-780; Wilson et al. (1994) J. Mol. Recog. 7: 89-98; Chen et al. (1995) Nucl. Acids Res 23: 2661-2668; Hirschbein et al. (1997) Antisense Nucl. Acid Drug Dev 7: 55-61). Merely to illustrate, the backbone of an RNAi construct can be modified with phosphorothioates, phosphoramidate, phosphodithioates, chimeric methylpbosphonate-phosphodiesters, peptide nucleic acids, 5-propynyl-pyrimidine containing oligomers or sugar modifications (e.g., 2′-substituted ribonucleosides, a-configuration). The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition. In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred. In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above.

Ribozymes molecules designed to catalytically cleave an mRNA transcripts can also be used to prevent translation of mRNA (see, e.g., PCT International Publication WO90/11364, published Oct. 4, 1990; Sarver et al., 1990, Science 247: 1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334: 585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224: 574-578; Zaug and Cech, 1986, Science, 231: 470-475; Zaug, et al., 1986, Nature, 324: 429-433; WO88/04300; Been and Cech, 1986, Cell, 47: 207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

Antibodies can be used as modulators of the activity of a particular protein. Antibodies can have extraordinary affinity and specificity for particular epitopes. Antibodies that bind to a particular protein in such a way that the binding of the antibody to the epitope on the protein can interfere with the function of that protein. For example, an antibody may inhibit the function of an ion channel by sterically hindering the proper ion channel subunits interactions or proper ion channel interactions with other molecules, or occupying active sites. Alternatively the binding of the antibody to an epitope on the particular protein may alter the conformation of that protein such that it is no longer able to properly function.

Monoclonal or polyclonal antibodies can be made using standard protocols (see, e.g., Antibodies: A Laboratory Manual ed. by Harlow and Lane (Cold Spring Harbor Press: 1988)). A mammal, such as a mouse, a hamster, a rat, a goat, or a rabbit can be immunized with an immunogenic form of the peptide. Techniques for conferring immunogenicity on a protein or peptide include conjugation to carriers or other techniques well known in the art.

Following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a particular polypeptide and monoclonal antibodies isolated from a culture comprising such hybridoma cells.

In the context of the present invention, antibodies can be screened and tested to identify those antibodies that can modulate the function of a particular ion channel. One of skill in the art will recognize that not every antibody that is specifically immunoreactive with a particular channel will affect the function of that channel. However, one of skill in the art can readily test antibodies to identify those that are capable of stimulating or blocking the function of a particular ion channel. Alternatively, screening assays of the invention may be used to test antibodies against a particular eukaryotic ion channel.

The term antibody as used herein is intended to include fragments thereof which are also specifically reactive with a particular ion channel. Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. The antibody of the present invention is further intended to include bispecific and chimeric molecules having affinity for a particular protein conferred by at least one CDR region of the antibody.

Both monoclonal and polyclonal antibodies (Ab) directed against a particular polypeptides, and antibody fragments such as Fab, F(ab)₂, Fv and scFv can be used to block the action of a particular protein. Such antibodies can be used either in an experimental context to further understand the role of a particular ion channel in a biological process, or in a therapeutic context.

Variants polypeptides and peptide fragments can agonize or antagonize the function of a particular protein. Examples of such variants and fragments include constitutively active or dominant negative mutants of a particular protein. Agonistic or antagonistic variants may function in any of a number of ways, for example, as described herein. One of skill in the art can readily make variants comprising an amino acid sequence at least 60%, 70%, 75%, 80%, 85%, 90%, 95%, 98% or 99% identical to a particular ion channel, or a fragment thereof, and identify variants that agonize or antagonize the function of the wild type channel protein.

Similarly, one can make peptide mimetics that agonize or antagonize the function of a particular protein. Methods of making various peptide mimetics are well known in the art, and one of skill can readily make a peptide mimetic of a particular ion channel, or fragment thereof, and identify mimetics that agonize or antagonize the function of the wild type channel protein.

Small organic molecules can agonize or antagonize the function of a particular protein. By small organic molecule is meant a carbon contain molecule having a molecular weight less than 2500 amu, more preferably less than 1500 amu, and even more preferably less than 750 amu.

Small organic molecules can be readily identified by screening libraries of organic molecules and/or chemical compounds to identify those compounds that have a desired function. Without being bound by theory, small organic molecules may exert their agonistic or inhibitory function in any of a number of ways. For example, the small molecule may promote or compete against an ion “binding” (or entry into the pore) to its channel. If a transporter is involved, the agent may compete for the ion binding site on that transporter. Similarly, the small organic molecule may bind to and alter the confirmation of the channel protein, and thus agonize or antagonize the function of that channel. In another example, the small organic molecules may bind to another site on the channel and potentiate or disrupt an interaction required for the functionality of the channel. To illustrate, a protein may require a protein, vitamin, metal, or other cofactor for functionality, and the small organic molecule may disrupt this interaction. For example, for a ligand-gated ion channel, a small molecule agent may potentiate or disrupt the ligand's action upon the ion channel.

In addition to small organic molecules, agents within the scope of the present invention include inorganic molecules. Inorganic molecules can be identified from amongst libraries of inorganic molecules or by selectively screening individual or pools of candidate agents, and can agonize or antagonize the activity of a membrane protein.

As will be readily apparent in light of the detailed description of exemplary agonistic and antagonistic agents described herein, the invention contemplates agents that modulate the activity of membrane proteins via any of a number of mechanisms. The screening methods described herein are not biased in favor of identifying agents that function through a particular mechanism, but are instead based on the identification of agents that alter (increase or decrease) one or more functional activity of titinic.

### Diseases, Disorders, or Conditions Related to Titinic Function

The present invention provides methods for screening to identify compounds that modulate one or more of the functional activities of a titinic voltage sensitive protein. Compounds identified as titinic modulators (either agonists or antagonists) may be useful in treating or developing treatments for diseases or conditions caused, in whole or in part, by misexpression or misregulation of a titinic protein.

Since titinic binds PKN1 in hyperlarized cells (but not depolarized) or cells in a resting state, titinic modulators should be useful for the treatment of central and peripheral nervous system conditions involving hyperexcitability of neurons such as epilepsy, pain, and cerebral ischemic conditions. Furthermore, given the expression pattern of titinic, one class of conditions that can be treated using the compounds identified in the methods of the present invention are inflammatory conditions and diseases caused, in whole or in part, via an effect on microglia. Exemplary conditions include inflammatory conditions and Alzheimer's disease (AD). Exemplary inflammatory diseases include, but are not limited to, neuroinflammatory diseases, asthma, chronic obstructive pulmonary disease, rheumatoid arthritis, osteoarthritis, inflammatory bowel disease, glomerulonephritis, multiple sclerosis, and disorders of the immune system.

In addition, the invention contemplates that numerous diseases may be caused or exacerbated by misregulation of proton transport, misregulation of superoxide ion production, misregulation of phosphatase activity in a cell, misregulation of intracellular pH, and the like. Because of the important role played by ion channel and membrane proteins in regulating cell signaling, gene expression, cell death, and homeostasis, compounds that agonize or antagonize one or more functions of titinic may be useful in understanding, treating, or preventing any of a number of disease or disorders. Exemplary diseases and disorders include dermatological diseases and disorders; neurological and neurodegenerative diseases and disorders; pain including nociceptive, inflammatory and neuropathic conditions, fever associated with various diseases, disorders or conditions; incontinence; inflammatory diseases and disorders such as inflammatory bowel disease and Crohn's disease; respiratory diseases and disorders such as chronic cough, asthma and chronic obstructive pulmonary disease (COPD); digestive disorders such as ulcers and acid reflux; metabolic diseases and disorders including obesity and diabetes; liver and kidney diseases and disorders; malignancies including cancers; and aging-related disorders.

This invention is based in part on the experiments described in the following examples. These examples are provided to illustrate the invention and should not be construed as limiting.

### Example 1

### Titinic is Highly Expressed in Central and Peripheral Nervous Tissues

Titinic tissue expression was determined as follows. A male rat weighing approximately 250 grams was sacrificed and organs were harvested. RNA was prepared using Tri-zol reagent. Briefly, tissues were snap-frozen and ground in a mortar and pestle and processed as directed by the manufacturer's protocol. cDNA was synthesized from the RNAs using SuperScript III and subsequently analyzed by semi-quantitative PCR (QPCR). QPCR was carried out on a Roche LightCycler 480. Two sets of gene specific primers designed against the rat titinic sequence were used with the Roche SYBR green kit. Item No.: 1

Name: rat titinic 170F
Sequence: TCGCAACAAGTAGACGAAGAAACC
Name: rat titinic 445R
Sequence: TTCCAGAGTCAGGAGAATCACCAC
Name: rat titinic 836F
Sequence: ACGCAAATCTGTCAGGAGCAAG
Name: rat titinic 1180R
Sequence: ATTGGGGTGGTGGATGTCTATG

Fluorescent signals for each tissue for the two sets of primers were averaged and normalized to the housekeeping gene, GAPDH (FIG. 5). Titinic expression was highest in brain and spinal cord, followed by the dorsal root ganglia and kidney.

### Example 2

### Titinic is Localized at the Cellular Plasma Membrane

The cellular localization of titinic was determined by examining cells transfected with a vector encoding titinic operably linked to GFP by confocal microscopy. As shown in FIG. 5, titinic localizes at the plasma membrane of cells.

### Example 3

### Titinic Binds PKN1 in Hyperpolarized but not Depolarized Cells

A yeast 2-hybrid was performed to identify proteins to which titinic binds. The human titinic protein was cloned into the pGBKT7 plasmid, which codes for a fusion of the bait protein with the binding domain of the GAL4 promoter. The human cDNA library uses the pGADT7 plasmid which codes for a fusion of GAL4's activation domain and a selection of library proteins. If Titinic interacts with a library protein, the GAL4 promoter will be activated. 127 positive clones were picked with 18 clones having β-galatosidase activity above background. The clone with the highest β-galactosidase activity was identified as a fragment of the serine/threonine kinase PKN1 (Accession numbers AAH40061 or AAH94766 and see, for example, Torbett et al., J. Biol. Chem. 278:32344-51, 2003). PKN1 is activated by rho GTPase, includes a PKC+ domain, and is activated by phospholipids.

Co-immunoprecipitation of PKN1 and titinic further verified that PKN1 interacts with titinic (FIG. 7). HEK293 cells expressing GFP, or a GFP-Titinic fusion protein were depolarised for 15 minutes with buffer containing 150 mM potassium chloride. Control cells were incubated in a 150 mM sodium chloride solution and were therefore not depolarized. Cells were subsequently lysed with buffer containing 1% NP40. Cell lysates were mixed with anti-GFP antibodies and the resulting complexes were precipitated using Protein A/G sepharose beads. The precipitated complexes were liberated from the beads with buffer containing 2% SDS and fractionated by denaturing polyacrylamide gel electrophoresis. The fractionated proteins were subjected to western blot analysis using an anti-PKN1 antibody (FIG. 7). Our results showing that titinic binds PKN1 in polarized state indicates that titinic binds to PKN1 in an activity dependent manner.

Our experimental studies have identified titinic, a membrane protein that contains a voltage sensitive domain and that is preferentially expressed in the central and nervous peripheral system (e.g., dorsal root ganglia, spinal cord, and brain). Our results showing that Titinic interacts with PKN1 in cells in a hyperpolarized but not depolarized state indicate a role for Titinic in central and peripheral nervous system conditions that involve the hyperexcitability of neurons. The identification of compounds having the ability to modulate titinic activity should therefore be useful for the treatment of such conditions.

SEQ ID NO: 1 (human titinic)
ATGGCTGTGGCTCCATCTTTCAACATGACCAATCCACAGCCTGCCATAGA
AGGAGGAATTTCTGAAGTTGAGATCATCTCCCAACAAGTAGACGAAGAA
ACCAAGAGCATTGCTCCTGTGCAGCTGGTGAACTTTGCCTATCGGGACTT
GCCCCTGGCTGCTGTCGATCTCTCCACGGCGGGCTCGCAGCTCCTGTCAA
ATCTGGACGAAGATTACCAAAGAGAAGGGTCTAACTGGCTGAAGCCGTG
CTGTGGGAAGAGAGCAGCCGTGTGGCAGGTATTTTTGCTCAGTGCAAGT
CTCAACAGTTTCCTGGTAGCCTGTGTAATATTGGTGGTGATTCTCCTGAC
TCTGGAACTTCTAATAGATATAAGCTTCTCCAGTTTTCCAGCGCATTCC
AGTTTGCTGGCGTGATTCACTGGATCAGCCTGGTCATTCTGTCCGTGTTC
TTCTCAGAGACTGTTCTACGGATTGTGGTGCTTGGGATCTGGGATTACAT
CGAAAACAAAATAGAGGTGTTTGACGGGGCTGTGATCATCCTATCTTTG
GCTCCGATGGTGGCATCCACTGTGGCCAAT
GGACCCAGGAGCCCCTGGGACGCCATCAGCCTCATCATCATGCTCCGGA
TCTGGAGGGTGAAGAGGGTCATTGATGCCTACGTCCTGCCAGTGAAGCT
GGAGATGGAGATGGTTATCCAGCAGTACGAGAAGGCCAAGGTCATCCAA
GACGAGCAGCTGGAGAGGCTGACGCAGATCTGTCAGGAGCAAGGGTTTG
AGATCCGGCAGCTGCGCGCGCACCTGGCGCAGCAGGACCTGGACCTGGC
TGCCGAGCGCGAAGCGGCGCTCCAGGCCCCGCACGTGCTCAGCCAGCCG
CGCAGCCGCTTCAAAGTGTTGGAGGCCGGCACGTGGGACGAGGAGACG
GCGGCCGAGAGCGTCGTG
GAGGAGCTGCAGCCCTCGCAAGAAGCCACGATGAAGGACGACATGAAC
AGCTACATCAGTCAGTATTACAATGGGCCCAGCAGTGACAGCGGTGTCC
CAGAGCCAGCTGTGTGTATGGTCACCACGGCCGCAATAGACATTCACCA
GCCCAACATCTCCTCGGACCTCTTCTCTCTGGACATGCCCCTCAAACTCG
GCGGTAATGGCACCAGCGCCACCTCGGAGAGTGCCTCCCGCAGCTCAGT
CACCCGGGCCCAGAGTGACAGCAGCCAGACGCTGGGCTCCTCCATGGAC
TGCAGCACTGCCCGCGAGGAGCCGTCCTCTGAGCCCGGCCCTTCTCCCCC
GCCGCTGCCATCCCAGCAGCAGGTGGAGGAGGCCACAGTCCAGGACCTG
CTGTCCTCCCTGTCGGAGGACCCCTGCCCTTCCCAGAAGGCCTTGGACCC
AGCCCCCCTCGCCCGGCCCAGCCCAGCGGGCTCGGCCCAAACCAGCCCC
GAGCTGGAACACAGGGTAAGTCTGTTCAACCAGAAGAACCAGGAGGGC
TTCACTGTCTTTCAGATCAGGCCTGTCATCCACTTCCAGCCGACTGTGCC
CATGCTGGAGGACAAGTTCAGATCTTTGGAATCCAAAGAGCAAAAGCTG
CACAGGGTCCCTGAGGCCTAG
SEQ ID NO: 2 (human titinic)
MAVAPSFNMTNPQPAIEGGISEVEIISQQVDEETKSIAPVQLVNFAYRDL
PLAAVDLSTAGSQLLSNLDEDYQREGSNWLKPCCGKRAAVWQVFLLSASL
NSFLVACVILVVILLTLELLIDIKLLQFSSAFQFAGVIHWISLVILSVFF
SETVLRIVVLGIWDYIENKIEVFDGAVIILSLAPMVASTVANGPRSPWDA
ISLIIMLRIWRVKRVIDAYVLPVKLEMEMVIQQYEKAKVIQDEQLERLTQ
ICQEQGFEIRQLRAHLAQQDLDLAAEREAALQAPHVLSQPRSRFKVLEAG
TWDEETAAESVVEELQPSQEATMKDDMNSYISQYYNGPSSDSGVPEPAVC
MVTTAAIDIHQPNISSDLFSLDMPLKLGGNGTSATSESASRSSVTRAQSD
SSQTLGSSMDCSTAREEPSSEPGPSPPPLPSQQQVEEATVQDLLSSLSED
PCPSQKALDPAPLARPSPAGSAQTSPELEHRVSLFNQKNQEGFTVFQIRP
VIHFQPTVPMLEDKFRSLESKEQKLHRVPEA
SEQ ID NO: 3 (rat titinic)
ATGGCCAGTCCACAACCTGCCATTGAAGGAGGGATTTCTGAAGTTGAGA
TTATCTCGCAACAAGTAGACGAAGAAACCAAGAACATTGCTCCGGTGCA
GCTGGTGAACTTTGCCTACCGGGACCTGCCCTTGGCTGCTGTAGACCTCT
CCACCGGGGGCTCACAGCTCCTGTCGAATTTGGACGAAGAGTACCAAAG
AGAAGGGTCTAACTGGCTGAAGCCGTGCTGTGGGAAGAGAGCGGCCGTG
TGGCAGGTACTTTTGCTCAGTGCAAGTCTCAACAGTTTCCTGGTAGCCTG
TGTAATATTGGTGGTGATTCTCCTGACTCTGGAACTTCTAATAGATATAA
AGCTTCTCCAGTTTTCCAGTGCATTCCAGTTTGCTGCTGTCATTCACTGG
ATCAGTCTGGTCATTCTCTCTGTGTTCTTCTCAGAGACTATTCTACGGAT
CGTGGTACTGGGGATCTGGGATTACATCGAAAACAAAATAGAGGTGTTCG
ATGGGGCTGTGATCATCCTGTCCTTGGCCCCGATGGTGGCGTCCACTGTG
GCTAACGGACCCAGGAGCCCCTGGGATGCCATCAGTCTCATTATCATGTT
CCGAATCTGGCGGGTGAAGAGGGTCATTGATGCCTATGTCCTGCCAGTCA
AGTTGGAGATGGAGATGGTCACCCAGCAGTACGAGAAGGCCAAGGCCA
TCCAAGACGAGCATCTGGAGAGACTGACGCAAATCTGTCAGGAGCAAGG
GTTTGAGATCCGGCAGCTGCGTGCGCACCTGGCACAGCAGGACCTGGAT
CTGGCAGCGGAGCGGGAGGCGGCGCTGCAGGCCCCACACGTGCTCAGCC
AGCCACGCAGCCGCTACAAGGTAGTGGAGGCTGGCACATGGGCCGAGG
AGACCGCAGCCGAGAGCATCATGGAGGAGCTGAGGCCCTCTCAAGAAG
CCATGGTGAAAGACGATATGAACAGCTACATCAGCCAATACTACAACGG
GCCCAGCAGTGACAGTGGAGCCCCAGAACCAGCAGTGTGTGTGGTCACT
ACAGCTGCCATAGACATCCACCAGCCCAATATCACCTCAGACCTCTTCTC
AGTCGACCTGCCTCTGAAGCTCAGTGGCAACAGCACCTGTGCCAGCGCC
ACCTCAGAGACCACCTCCCACCCCACCTGTGGCTCAGTCACCAGGGCCC
AGAGTGCCAGCAGCCAGACACTGGGTTCCTCCACAGACTGTAGCACCCC
CCGAGAAGAGCTGTCCTCTAAACCCATATCTTCTCCCCTGCCACTGCTTC
TGCCCCCTCAGCAGCTGGTGGCGGAGGCCACAGTCCAGGACCTGATGTC
CTCTCTGTCAAAGGACCCCTGTCCATCCCATAAGGCCTTGGACCCAGCAC
CCCTGGCCCAGCCTACTCCAGTGGGCTCTGTCCAGACCAGCCCTGAGCTG
GAACATAGGGTAAGTCTGTTCAACCAGAAGAACGAGGAGGCCGTCCCTG
TTCTTCAGATCAAGCCTGTCATCCACTTGCAGCCCACAGCTGGGCTGGAG
GAGAAGTTCAGATCTTTGGAATCCAAAGAGCCAAAGTTGCATACGGTTC
CTGAGACCTAG
SEQ ID NO: 4 (rat titinic)
MASPQPAIEGGISEVEIISQQVDEETKNIAPVQLVNFAYRDLPLAAVDLS
TGGSQLLSNLDEEYQREGSNWLKPCCGKRAAVWQVLLLSASLNSFLVACV
ILVVILLTLELLIDIKLLQFSSAFQFAAVIHWISLVILSVFFSETILRIV
VLGIWDYIENKIEVFDGAVIILSLAPMVASTVANGPRSPWDAISLIIMFR
IWRVKRVIDAYVLPVKLEMEMVTQQYEKAKAIQDEHLERLTQICQEQGFE
IRQLRAHLAQQDLDLAAEREAALQAPHVLSQPRSRYKVVEAGTWAEETAA
ESIMEELRPSQEAMVKDDMNSYISQYYNGPSSDSGAPEPAVCVVTTAAID
IHQPNITSDLFSVDLPLKLSGNSTCASATSETTSHPTCGSVTRAQSASSQ
TLGSSTDCSTPREELSSKPISSPLPLLLPPQQLVAEATVQDLMSSLSKDP
CPSHKALDPAPLAQPTPVGSVQTSPELEHRVSLFNQKNQEALPVLQIKPV
LHLQPTAGLEEKFRSLESKEPKLHTVPET.
SEQ ID NO: 5 (Mouse cDNA)
ATGGCTTTGGTTACATCTTTCAACATGGCCAATCCACAACCTGCCATTGA
AGGAGGAATTTCTGAAGTTGAGATTATCTCCCAACAAGTAGACGAAGAA
ACCAAGAGCATTGCTCCGGTGCAGCTGGTGAACTTTGCCTATCGGGACCT
GCCCCTGGCTGCCGTAGACCTCTCCACAGGGGGCTCACAGCTCCTGTCGA
ATTTGGACGAAGAGTACCAAAGAGAAGGGTCTGACTGGCTGAAGCCGTG
CTGTGGGAAGAGAGCAGCCGTATGGCAGGTATTTTTGCTCAGTGCAAGT
CTCAACAGTTTCCTGGTAGCCTGTGTAATATTGGTGGTGATCCTCCTGAC
TCTGGAGCTTCTCATAGATACAAAGCTTCTCCAGTTTTCCAATGCTTTCC
AGTTTGCTGGTGTCATTCACTGGATCAGTCTGGTCATTCTCTCTGTGTTC
TTCTCAGAGACTGTCCTACGGATCGTGGTACTGGGGATCTGGGATTACAT
CGAAAACAAAATAGAGGTGTTTGACGGGGCTGTGATCATCCTGTCCTTGG
CCCCGATGGTGGCGTCCACTGTGGCTAACGGACCCAGGAGCCCCTGGGA
TGCCATCAGCCTCATCATCATGTTCCGAATCTGGCGGGTGAAGAGGGTC
ATTGATGCCTATGTCCTGCCAGTCAAGTTGGAGATGGAGATGGTCACCC
AGCAGTATGAGAAGGCCAAGGCCATCCAAGATGAGCAGCTGGAAAGAC
TGACGCAAATCTGTCAGGAGCAAGGGTTTGAGATCCGGCAGCTGCGTGC
GCACCTGGCACAGCAGGACCTGGATCTGGCAGCCGAGCGGGAGGCGGC
GCTGCAGGCCCCACACGTGCTCAGCCAGCCACGCAGCCGCTACAAGGTC
GTAGAGGCTGGCACGTGGGCCGAGGAGACAGCAGCCGAGAGCATCGTG
GAAGAGCTGAGGCCCTCTCAAGAAGCCACAGTGAAAGATGACATGAAC
AGCTACATCAGCCAATACTACAATGGGCCCAGCAGTGACAGTGGAGCCC
CAGAACCAGCAGTATGTGTGGTCACTACAGCTGCCATAGACATCCACCA
GCCCAATGTCCCCTCAGACCTCTTCTCAGTCGACCTGCCTCTGAAGCTCA
GTGGCAACAGCACCTGTGCCAGCGCCACCTCGGAGACCACCTCCCACTC
TACCTGTGGCTCAGTCACCAGGGCCCAGAGTGCCAGCAGCCAGACACTG
GGTTCCTCCACAGACTGTAGCACCCCCCGGGAAGAGCTGCTGCCCTCTA
AGCCCAGATCTTCTCCCCTGCCACTGCTTCTGCCCCCTCAGCAGCTGGTG
GCAGAGGCCACAGTCCAGGACCTGATGTCCTCTCTGTCAAAGGACCCCT
GCCCATCCCATAAGGCCTTGGACCCAGCACCCCTGGCCCAGCCTACCCC
ACTGGGCTCAGTCCAGACCAGCCCTGAGCTGGAGCATAGGGTGAGTCTG
TTCAACCAGAAGAACCAGGAGGCTGTCCCTGTTCTTCAGATCAACCCTGT
CATCCACTTGCAGCCCACAGCGGGGCTGGAGGAGAAGTTCAGATCTTTG
GAATCCAAAGAGCCAAAGTTGCATACAGTTCCTGAGGCCTAG
SEQ ID NO:6 Mouse Protein
MALVTSFNMANPQPAIEGGISEVEIISQQVDEETKSIAPVQLVNFAYRDL
PLAAVDLSTGGSQLLSNLDEEYQREGSDWLKPCCGKRAAVWQVFLLSASL
NSFLVACVILVVILLTLELLIDTKLLQFSNAFQFAGVIHWISLVILSVFF
SETVLRIVVLGIWDYIENKIEVFDGAVIILSLAPMVASTVANGPRSPWDA
ISLIIMFRIWRVKRVIDAYVLPVKLEMEMVTQQYEKAKAIQDEQLERLTQ
ICQEQGFEIRQLRAHLAQQDLDLAAEREAALQAPHVLSQPRSRYKVVEAG
TWAEETANESIVEELRPSQEATVKDDMNSYISQYYNGPSSDSGAPEPAVC
VVTTAAIDIHQPNVPSDLFSVDLPLKLSGNSTCASATSETTSHSTCGSVT
RAQSASSQTLGSSTDCSTPREELLPSKPRSSPLPLLLPPQQLVAEATVQD
LMSSLSKDPCPSHKALDPAPLAQPTPLGSVQTSPELEHRVSLFNQKNQEA
LPVLQINPVIHLQPTAGLEEKFRSLESKEPKLHTVPEA
SEQ ID NO: 7: Human Sequence (with UTR)
AGAACCCACGCTTGGAAATGCTGACAGCAGGCTTCAGGACAGCTGAGCC
CCACTAAACACCAAGAAAACCCATGGCTGTGGCTCCATCTTTCAACATG
ACCAATCCACAGCCTGCCATAGAAGGAGGAATTTCTGAAGTTGAGATCA
TCTCCCAACAAGTAGAGGAAGAAACCAAGAGCATTGCTCCTGTGCAGCT
GGTGAACTTTGCCTATCGGGACTTGCCCCTGGCTGCTGTCGATCTCTCCA
CGGCGGGCTCGCAGCTCCTGTCAAATCTGGACGAAGATTACCAAAGAGA
AGGGTCTAACTGGCTGAAGCCGTGCTGTGGGAAGAGAGCAGCCGTGTGG
CAGGTATTTTTGCTCAGTGCAAGTCTCAACAGTTTCCTGGTAGCCTGTGT
AATATTGGTGGTGATTCTCCTGACTCTGGAACTTCTAATAGATATAAAGC
TTCTCCAGTTTTCCAGCGCATTCCAGTTTGCTGGCGTGATTCACTGGATC
AGCCTGGTCATTCTGTCCGTGTTCTTCTCAGAGACTGTTCTACGGATTGT
GGTGCTTGGGATCTGGGATTACATCGAAAACA
AAATAGAGGTGTTTGACGGGGCTGTGATCATCCTATCTTTGGCTCCGATG
GTGGCATCCACTGTGGCCAATGGACCCAGGAGCCCCTGGGACGCCATCA
GCCTCATCATCATGCTCCGGATCTGGAGGGTGAAGAGGGTCATTGATGC
CTACGTCCTGCCAGTGAAGCTGGAGATGGAGATGGTTATCCAGCAGTAC
GAGAAGGCCAAGGTCATCCAAGACGAGCAGCTGGAGAGGCTGACGCAG
ATCTGTCAGGAGCAAGGGTTTGAGATCCGGCAGCTGCGCGCGCACCTGG
CGCAGCAGGACCTGGACCTGGCTGCCGAGCGCGAAGCGGCGCTCCAGGC
CCCGCACGTGCTCAGCC
AGCCGCGCAGCCGCTTCAAAGTGTTGGAGGCCGGCACGTGGGACGAGGA
GACGGCGGCCGAGAGCGTCGTGGAGGAGCTGCAGCCCTCGCAAGAAGC
CACGATGAAGGACGACATGAACAGCTACATCAGTCAGTATTACAATGGG
CCCAGCAGTGACAGCGGTGTCCCAGAGCCAGCTGTGTGTATGGTCACCA
CGGCCGCAATAGACATTCACCAGCCCAACATCTCCTCGGACCTCTTCTCT
CTGGACATGCCCCTCAAACTCGGCGGTAATGGCACCAGCGCCACCTCGG
AGAGTGCCTCCCGCAGCTCAGTCACCCGGGCCCAGAGTGACAGCAGCCA
GACGCTGGGCTCCTCCA
TGGACTGCAGCACTGCCCGCGAGGAGCCGTCCTCTGAGCCCGGCCCTTCT
CCCCCGCCGCTGCCATCCCAGCAGCAGGTGGAGGAGGCCACAGTCCAGG
ACCTGCTGTCCTCCCTGTCGGAGGACCCCTGCCCTTCCCAGAAGGCCTTG
GACCCAGCCCCCCTCGCCCGGCCCAGCCCAGCGGGCTCGGCCCAAACCA
GCCCCGAGCTGGAACACAGGGTAAGTCTGTTCAACCAGAAGAACCAGGA
GGGCTTCACTGTCTTTCAGATCAGGCCTGTCATCCACTTCCAGCCCACTG
TGCCCATGCTGGAGGACAAGTTCAGATCTTTGGAATCCAAAGAGCAAAA
GCTGCACAGGGTCCCTGAGGCCTAGAGCCTGCCATGGGCTGGGTGAGAT
GAGGGGAGACAGCCATCTCAAAGCTCTCCTGGGACCCTG

### Incorporation by Reference

All publications and patents mentioned herein, are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An isolated nucleic acid encoding a titinic protein, wherein the nucleic acid comprises a nucleotide sequence that hybridizes under stringent conditions, including a wash step of 0.2×SSC at 65° C., to the nucleic acid sequence that is complementary to the sequence set forth in SEQ ID NO: 1.

2. The isolated nucleic acid of claim 1, wherein the nucleic acid comprises a nucleotide sequence set for in SEQ ID NO: 1.

3. An isolated nucleic acid encoding a titinic protein, wherein the titinic protein comprises a four transmembrane domain containing voltage sensitive protein, and wherein the titinic protein comprises an amino acid sequence at least 90% identical to SEQ ID NO: 2.

4. The isolated nucleic acid of claim 3, wherein the protein comprises an amino acid sequence at least 95% identical to SEQ ID NO: 2.

5. The isolated nucleic acid of claim 4, wherein the protein comprises an amino acid sequence 100% identical to SEQ ID NO: 2.

6. A nucleic acid composition comprising:
a nucleic acid encoding a titinic protein, wherein the titinic protein comprises a four transmembrane domain containing voltage sensitive protein, and wherein the nucleic acid comprises a nucleotide sequence that hybridizes under stringent conditions, including a wash step of 0.2×SSC at 65° C., to the nucleic acid sequence that is complementary to the sequence set forth in SEQ ID NO: 1; and
a heterologous nucleic acid sequence.

7. An expression vector, which replicates in at least one of a prokaryotic cell and eukaryotic cell, comprising the nucleic acid of claim 1 or claim 6.

8. A host cell transfected with the expression vector of claim 7 and expressing said protein.

9. The host cell of claim 8, wherein said cell is a bacterial cell.

10. The host cell of claim 8, wherein said cell is a CHO cell or an HEK cell.

11. The host cell of claim 8, wherein said cell is a neuronal cell type.

12. A method of producing a recombinant titinic protein comprising culturing the cell of claim 8 in a cell culture medium to express said titinic protein;
expressing said protein; and
isolating said protein from said cell culture.

13. An in vitro recombinant transfection system, comprising a gene construct including the nucleic acid of claim 1 or 6 operably linked to a transcriptional regulatory sequence for causing expression of the titinic protein in prokaryotic or eukaryotic cells; and
a gene delivery composition for delivering said gene construct to a cell and causing the cell to be transfected with said gene construct.

14. The recombinant transfection system of claim 13, wherein the transcriptional regulatory sequence is a conditional transcriptional regulatory sequence.

15. A nucleic acid comprising:
a nucleotide sequence encoding a polypeptide comprising a titinic protein, wherein said nucleotide sequence hybridizes under stringent conditions, including a wash step of 0.2×SSC at 65° C., to a nucleic acid sequence that is complementary to the sequence set forth in SEQ ID NO: 1; and
a transcriptional regulatory sequence operably linked to said coding sequence and heterologous thereto.

16. An isolated or recombinantly produced protein, comprising an amino acid sequence at least 90% identical to SEQ ID NO: 2, wherein the protein is a titinic protein comprising four transmembrane domains.

17. The isolated or recombinantly produced protein of claim 16, wherein the protein comprises an amino acid sequence at least 95% identical to SEQ ID NO: 2.

18. The isolated or recombinantly produced protein of claim 17, wherein the protein comprises an amino acid sequence identical to SEQ ID NO: 2.

19. An isolated or recombinantly produced protein, comprising an amino acid sequence encoded by a nucleic acid sequence that hybridizes under stringent conditions, including a wash step of 0.2×SSC at 65° C., to a sequence that is complementary to SEQ ID NO: 1, wherein the protein is a titinic voltage sensitive protein comprising four transmembrane domains.

20. A method of screening for compounds that modulate an activity of a titinic protein, comprising:
providing a cell expressing a titinic protein and a PKN1 protein;
contacting said cell with a candidate compound;
detecting a change in PKN1 activity in said cell in the presence of said compound versus the absence of said compound,
wherein a compound that promotes a change in PKN1 activity is a compound that modulates an activity of a titinic protein.

21. The method of claim 20, wherein the titinic protein comprises a four transmembrane domain protein encoded by a nucleic acid that hybridizes under stringent conditions, including a wash step of 0.2×SSC at 65° C., to a nucleic acid sequence that is complementary to the sequence set forth in SEQ ID NO: 1.

22. The method of claim 20, wherein the cell comprises a vector comprising a nucleic acid sequence encoding the titinic protein.

23. The method of claim 20, wherein the cell is a bacterial cell.

24. The method of claim 20, where the cell is an HEK cell or a CHO cell.

25. The method of claim 24, wherein the HEK cell or CHO cell comprises a vector comprising a nucleic acid sequence encoding the titinic protein.

26. The method of claim 20, wherein the cell is a neuronal cell.

27. The method of claim 26, wherein the neuronal cell comprises a vector comprising a nucleic acid sequence encoding the titinic protein.

28. The method of claim 20, wherein the compound agonizes the activity of the titinic protein.

29. The method of claim 20, wherein the compound antagonizes the activity of the titinic protein.

30. The method of claim 20, wherein the compound binds to the titinic protein.

31. The method of claim 20, wherein the change in PKN1 activity is a decrease in PKN1 activity.

32. The method of claim 20, wherein the change in PKN1 activity is an increase in PKN1 activity.

33. The method of claim 20, wherein detecting a change in PKN1 activity comprises detecting a change in fluorescence of a fluorescent indicator protein operably linked to the PKN1 protein.

34. The method of claim 20, wherein said cell is a depolarized cell.

35. The method of claim 20, wherein the compound is a small molecule with a molecular weight of less than approximately 600 daltons.

36. An isolated or recombinantly produced antibody immunoreactive with a titinic protein, wherein the titinic protein comprises an amino acid sequence at least 90% identical to SEQ ID NO: 2.


SEQUENCE LISTING (7 sequences)

SEQ ID NO: 1 - DNA, 1596 bp, Homo sapiens
atggctgtggctccatctttcaacatgaccaatccacagcctgccatagaaggaggaatttctgaagttgagatcatctcccaacaagtagacgaagaaaccaagagcattgctcctgtgcagctggtgaactttgcctatcgggacttgcccctggctgctgtcgatctctccacggcgggctcgcagctcctgtcaaatctggacgaagattaccaaagagaagggtctaactggctgaagccgtgctgtgggaagagagcagccgtgtggcaggtatttttgctcagtgcaagtctcaacagtttcctggtagcctgtgtaatattggtggtgattctcctgactctggaacttctaatagatataaagcttctccagttttccagcgcattccagtttgctggcgtgattcactggatcagcctggtcattctgtccgtgttcttctcagagactgttctacggattgtggtgcttgggatctgggattacatcgaaaacaaaatagaggtgtttgacggggctgtgatcatcctatctttggctccgatggtggcatccactgtggccaatggacccaggagcccctgggacgccatcagcctcatcatcatgctccggatctggagggtgaagagggtcattgatgcctacgtcctgccagtgaagctggagatggagatggttatccagcagtacgagaaggccaaggtcatccaagacgagcagctggagaggctgacgcagatctgtcaggagcaagggtttgagatccggcagctgcgcgcgcacctggcgcagcaggacctggacctggctgccgagcgcgaagcggcgctccaggccccgcacgtgctcagccagccgcgcagccgcttcaaagtgttggaggccggcacgtgggacgaggagacggcggccgagagcgtcgtggaggagctgcagccctcgcaagaagccacgatgaaggacgacatgaacagctacatcagtcagtattacaatgggcccagcagtgacagcggtgtcccagagccagctgtgtgtatggtcaccacggccgcaatagacattcaccagcccaacatctcctcggacctcttctctctggacatgcccctcaaactcggcggtaatggcaccagcgccacctcggagagtgcctcccgcagctcagtcacccgggcccagagtgacagcagccagacgctgggctcctccatggactgcagcactgcccgcgaggagccgtcctctgagcccggcccttctcccccgccgctgccatcccagcagcaggtggaggaggccacagtccaggacctgctgtcctccctgtcggaggacccctgcccttcccagaaggccttggacccagcccccctcgcccggcccagcccagcgggctcggcccaaaccagccccgagctggaacacagggtaagtctgttcaaccagaagaaccaggagggcttcactgtctttcagatcaggcctgtcatccacttccagcccactgtgcccatgctggaggacaagttcagatctttggaatccaaagagcaaaagctgcacagggtccctgaggcctag

SEQ ID NO: 2 - protein, 531 aa, Homo sapiens
MAVAPSFNMTNPQPAIEGGISEVEIISQQVDEETKSIAPVQLVNFAYRDLPLAAVDLSTAGSQLLSNLDEDYQREGSNWLKPCCGKRAAVWQVFLLSASLNSFLVACVILVVILLTLELLIDIKLLQFSSAFQFAGVIHWISLVILSVFFSETVLRIVVLGIWDYIENKIEVFDGAVIILSLAPMVASTVANGPRSPWDAISLIIMLRIWRVKRVIDAYVLPVKLEMEMVIQQYEKAKVIQDEQLERLTQICQEQGFEIRQLRAHLAQQDLDLAAEREAALQAPHVLSQPRSRFKVLEAGTWDEETAAESVVEELQPSQEATMKDDMNSYISQYYNGPSSDSGVPEPAVCMVTTAAIDIHQPNISSDLFSLDMPLKLGGNGTSATSESASRSSVTRAQSDSSQTLGSSMDCSTAREEPSSEPGPSPPPLPSQQQVEEATVQDLLSSLSEDPCPSQKALDPAPLARPSPAGSAQTSPELEHRVSLFNQKNQEGFTVFQIRPVIHFQPTVPMLEDKFRSLESKEQKLHRVPEA

SEQ ID NO: 3 - DNA, 1590 bp, Rat
atggccagtccacaacctgccattgaaggagggatttctgaagttgagattatctcgcaacaagtagacgaagaaaccaagaacattgctccggtgcagctggtgaactttgcctaccgggacctgcccttggctgctgtagacctctccaccgggggctcacagctcctgtcgaatttggacgaagagtaccaaagagaagggtctaactggctgaagccgtgctgtgggaagagagcggccgtgtggcaggtacttttgctcagtgcaagtctcaacagtttcctggtagcctgtgtaatattggtggtgattctcctgactctggaacttctaatagatataaagcttctccagttttccagtgcattccagtttgctgctgtcattcactggatcagtctggtcattctctctgtgttcttctcagagactattctacggatcgtggtactggggatctgggattacatcgaaaacaaaatagaggtgttcgatggggctgtgatcatcctgtccttggccccgatggtggcgtccactgtggctaacggacccaggagcccctgggatgccatcagtctcattatcatgttccgaatctggcgggtgaagagggtcattgatgcctatgtcctgccagtcaagttggagatggagatggtcacccagcagtacgagaaggccaaggccatccaagacgagcatctggagagactgacgcaaatctgtcaggagcaagggtttgagatccggcagctgcgtgcgcacctggcacagcaggacctggatctggcagcggagcgggaggcggcgctgcaggccccacacgtgctcagccagccacgcagccgctacaaggtagtggaggctggcacatgggccgaggagaccgcagccgagagcatcatggaggagctgaggccctctcaagaagccatggtgaaagacgatatgaacagctacatcagccaatactacaacgggcccagcagtgacagtggagccccagaaccagcagtgtgtgtggtcactacagctgccatagacatccaccagcccaatatcacctcagacctcttctcagtcgacctgcctctgaagctcagtggcaacagcacctgtgccagcgccacctcagagaccacctcccaccccacctgtggctcagtcaccagggcccagagtgccagcagccagacactgggttcctccacagactgtagcaccccccgagaagagctgtcctctaaacccatatcttctcccctgccactgcttctgccccctcagcagctggtggcggaggccacagtccaggacctgatgtcctctctgtcaaaggacccctgtccatcccataaggccttggacccagcacccctggcccagcctactccagtgggctctgtccagaccagccctgagctggaacatagggtaagtctgttcaaccagaagaaccaggaggccctccctgttcttcagatcaagcctgtcatccacttgcagcccacagctgggctggaggagaagttcagatctttggaatccaaagagccaaagttgcatacggttcctgagacctag

SEQ ID NO: 4 - protein, 529 aa, Rat
MASPQPAIEGGISEVEIISQQVDEETKNIAPVQLVNFAYRDLPLAAVDLSTGGSQLLSNLDEEYQREGSNWLKPCCGKRAAVWQVLLLSASLNSFLVACVILVVILLTLELLIDIKLLQFSSAFQFAAVIHWISLVILSVFFSETILRIVVLGIWDYIENKIEVFDGAVIILSLAPMVASTVANGPRSPWDAISLIIMFRIWRVKRVIDAYVLPVKLEMEMVTQQYEKAKAIQDEHLERLTQICQEQGFEIRQLRAHLAQQDLDLAAEREAALQAPHVLSQPRSRYKVVEAGTWAEETAAESIMEELRPSQEAMVKDDMNSYISQYYNGPSSDSGAPEPAVCVVTTAAIDIHQPNITSDLFSVDLPLKLSGNSTCASATSETTSHPTCGSVTRAQSASSQTLGSSTDCSTPREELSSKPISSPLPLLLPPQQLVAEATVQDLMSSLSKDPCPSHKALDPAPLAQPTPVGSVQTSPELEHRVSLFNQKNQEALPVLQIKPVIHLQPTAGLEEKFRSLESKEPKLHTVPET

SEQ ID NO: 5 - DNA, 1617 bp, Mouse
atggctttggttacatctttcaacatggccaatccacaacctgccattgaaggaggaatttctgaagttgagattatctcccaacaagtagacgaagaaaccaagagcattgctccggtgcagctggtgaactttgcctatcgggacctgcccctggctgccgtagacctctccacagggggctcacagctcctgtcgaatttggacgaagagtaccaaagagaagggtctgactggctgaagccgtgctgtgggaagagagcagccgtatggcaggtatttttgctcagtgcaagtctcaacagtttcctggtagcctgtgtaatattggtggtgatcctcctgactctggagcttctcatagatacaaagcttctccagttttccaatgctttccagtttgctggtgtcattcactggatcagtctggtcattctctctgtgttcttctcagagactgtcctacggatcgtggtactggggatctgggattacatcgaaaacaaaatagaggtgtttgacggggctgtgatcatcctgtccttggccccgatggtggcgtccactgtggctaacggacccaggagcccctgggatgccatcagcctcatcatcatgttccgaatctggcgggtgaagagggtcattgatgcctatgtcctgccagtcaagttggagatggagatggtcacccagcagtatgagaaggccaaggccatccaagatgagcagctggaaagactgacgcaaatctgtcaggagcaagggtttgagatccggcagctgcgtgcgcacctggcacagcaggacctggatctggcagccgagcgggaggcggcgctgcaggccccacacgtgctcagccagccacgcagccgctacaaggtcgtagaggctggcacgtgggccgaggagacagcagccgagagcatcgtggaagagctgaggccctctcaagaagccacagtgaaagatgacatgaacagctacatcagccaatactacaatgggcccagcagtgacagtggagccccagaaccagcagtatgtgtggtcactacagctgccatagacatccaccagcccaatgtcccctcagacctcttctcagtcgacctgcctctgaagctcagtggcaacagcacctgtgccagcgccacctcggagaccacctcccactctacctgtggctcagtcaccagggcccagagtgccagcagccagacactgggttcctccacagactgtagcaccccccgggaagagctgctgccctctaagcccagatcttctcccctgccactgcttctgccccctcagcagctggtggcagaggccacagtccaggacctgatgtcctctctgtcaaaggacccctgcccatcccataaggccttggacccagcacccctggcccagcctaccccactgggctcagtccagaccagccctgagctggagcatagggtgagtctgttcaaccagaagaaccaggaggctctccctgttcttcagatcaaccctgtcatccacttgcagcccacagcggggctggaggagaagttcagatctttggaatccaaagagccaaagttgcatacagttcctgaggcctag

SEQ ID NO: 6 - protein, 538 aa, Mouse
MALVTSFNMANPQPAIEGGISEVEIISQQVDEETKSIAPVQLVNFAYRDLPLAAVDLSTGGSQLLSNLDEEYQREGSDWLKPCCGKRAAVWQVFLLSASLNSFLVACVILVVILLTLELLIDTKLLQFSNAFQFAGVIHWISLVILSVFFSETVLRIVVLGIWDYIENKIEVFDGAVIILSLAPMVASTVANGPRSPWDAISLIIMFRIWRVKRVIDAYVLPVKLEMEMVTQQYEKAKAIQDEQLERLTQICQEQGFEIRQLRAHLAQQDLDLAAEREAALQAPHVLSQPRSRYKVVEAGTWAEETAAESIVEELRPSQEATVKDDMNSYISQYYNGPSSDSGAPEPAVCVVTTAAIDIHQPNVPSDLFSVDLPLKLSGNSTCASATSETTSHSTCGSVTRAQSASSQTLGSSTDCSTPREELLPSKPRSSPLPLLLPPQQLVAEATVQDLMSSLSKDPCPSHKALDPAPLAQPTPLGSVQTSPELEHRVSLFNQKNQEALPVLQINPVIHLQPTAGLEEKFRSLESKEPKLHTVPEA

SEQ ID NO: 7 - DNA, 1730 bp, Homo sapiens
agaacccacgcttggaaatgctgacagcaggcttcaggacagctgagccccactaaacaccaagaaaacccatggctgtggctccatctttcaacatgaccaatccacagcctgccatagaaggaggaatttctgaagttgagatcatctcccaacaagtagacgaagaaaccaagagcattgctcctgtgcagctggtgaactttgcctatcgggacttgcccctggctgctgtcgatctctccacggcgggctcgcagctcctgtcaaatctggacgaagattaccaaagagaagggtctaactggctgaagccgtgctgtgggaagagagcagccgtgtggcaggtatttttgctcagtgcaagtctcaacagtttcctggtagcctgtgtaatattggtggtgattctcctgactctggaacttctaatagatataaagcttctccagttttccagcgcattccagtttgctggcgtgattcactggatcagcctggtcattctgtccgtgttcttctcagagactgttctacggattgtggtgcttgggatctgggattacatcgaaaacaaaatagaggtgtttgacggggctgtgatcatcctatctttggctccgatggtggcatccactgtggccaatggacccaggagcccctgggacgccatcagcctcatcatcatgctccggatctggagggtgaagagggtcattgatgcctacgtcctgccagtgaagctggagatggagatggttatccagcagtacgagaaggccaaggtcatccaagacgagcagctggagaggctgacgcagatctgtcaggagcaagggtttgagatccggcagctgcgcgcgcacctggcgcagcaggacctggacctggctgccgagcgcgaagcggcgctccaggccccgcacgtgctcagccagccgcgcagccgcttcaaagtgttggaggccggcacgtgggacgaggagacggcggccgagagcgtcgtggaggagctgcagccctcgcaagaagccacgatgaaggacgacatgaacagctacatcagtcagtattacaatgggcccagcagtgacagcggtgtcccagagccagctgtgtgtatggtcaccacggccgcaatagacattcaccagcccaacatctcctcggacctcttctctctggacatgcccctcaaactcggcggtaatggcaccagcgccacctcggagagtgcctcccgcagctcagtcacccgggcccagagtgacagcagccagacgctgggctcctccatggactgcagcactgcccgcgaggagccgtcctctgagcccggcccttctcccccgccgctgccatcccagcagcaggtggaggaggccacagtccaggacctgctgtcctccctgtcggaggacccctgcccttcccagaaggccttggacccagcccccctcgcccggcccagcccagcgggctcggcccaaaccagccccgagctggaacacagggtaagtctgttcaaccagaagaaccaggagggcttcactgtctttcagatcaggcctgtcatccacttccagcccactgtgcccatgctggaggacaagttcagatctttggaatccaaagagcaaaagctgcacagggtccctgaggcctagagcctgccatgggctgggtgagatgaggggagacagccatctcaaagctctcctgggaccctg